(19) United States
(12) Patent Application Publication
(10) Pub. No.: US 2024/0269459 A1
(43) Pub. Date: Aug. 15, 2024

(54) HEART PUMP IMPLANT SYSTEM WITH FASTENING AND RELEASING DEVICES

(71) Applicant: KARDION GMBH, Stuttgart (DE)

(72) Inventors: Inga Schellenberg, Stuttgart (DE); Hardy Baumbach, Stuttgart (DE); Mario Heintze, Stuttgart (DE); Thomas Müller, Stuttgart (DE); Leon Wenning, Stuttgart (DE)

(21) Appl. No.: 18/645,778

(22) Filed: Apr. 25, 2024

Related U.S. Application Data
(63) Continuation of application No. PCT/US2022/078609, filed on Oct. 24, 2022.
(60) Provisional application No. 63/263,043, filed on Oct. 26, 2021.

(51) Int. Cl. A61M 60/867; A61M 60/13; A61M 60/139; A61M 60/216; A61M 60/414; A61M 60/90

(52) U.S. Cl. CPC A61M 60/867; A61M 60/13; A61M 60/139; A61M 60/216; A61M 60/414; A61M 60/90; A61M 2205/0216; A61M 2205/025; A61M 2205/0266; A61M 2205/04; A61M 2205/103; A61M 2205/32; A61M 2210/125; A61M 2210/127

(57) ABSTRACT

Disclosed herein are systems and methods relating to an implant device, such as a heart pump. The implant device may comprise an implant, a fastening device, a release device, and a transfer device. The implant may be shaped for implantation in a vascular canal. The fastening device may have a coupling section that is coupled to the implant and is movable between a fastening position, in which the fastening device is configured to fasten the implant in the vascular canal, and a release device, which may be configured to transfer the fastening device to a release position and releases the implant. The transfer device may be coupled to the fastening device and is adapted to cause transfer of the fastening device between the fastening position and the release position in response to an actuation.

### INCORPORATION BY REFERENCE

The present application is a continuation of International Application No. PCT/US2022/078609, filed Oct. 24, 2022, titled HEART PUMP IMPLANT SYSTEM WITH FASTENING AND RELEASING DEVICES, which claims the benefit of U.S. Provisional Application No. 63/263,043, filed Oct. 26, 2021, titled IMPLANT DEVICE, OPERATING DEVICE FOR OPERATING AN IMPLANT DEVICE, IMPLANT SYSTEM AND METHOD FOR MANUFACTURING AN IMPLANT DEVICE, the entire contents of each of which are incorporated by reference herein and made a part of this specification for all purposes.

### FIELD

The present disclosure relates generally to medical implants, in particular to devices, processes, and computer programs for performing minimally invasive delivery and implantation of heart pumps.

### BACKGROUND

Minimally invasive delivery systems in medical technology are often designed with regard to various factors such as function, operating principle, design, safety, ergonomics, production, control and testing, assembly, transport and/or use. These factors can likewise be considered during the design of anchoring mechanisms for anchoring an implant in a vascular system. Resulting solution variants are evaluated with the help of these factors, whereby each factor can have a specific weighting.

With regard to this evaluation, a wide variety of handle designs are conceived and designed. The design of handles, also referred to in the following as operating equipment, as well as specific mechanisms must therefore be individually adapted to the given requirements of the product.

Similarly, a wide variety of different anchoring mechanisms for anchoring an implant in a vascular system is available on the market. Self-expanding stent systems made of Nitinol are used in a variety of applications in medical technology to prevent migration of the implant by applying a radial clamping force against the inner wall of the vessel.

### SUMMARY

Disclosed herein are systems and methods relating to an implant device which can include any combination of the following: a fastening device for fastening an implant in a vascular canal, a release device for releasing an implant into a vascular canal, and an actuator for implanting an implant into a vascular canal. The approach presented here further may include an operating device for operating an implant device, an implant system, a method for manufacturing an implant device, a device using this method, and finally a corresponding computer program in accordance with the main requirements. By the measures listed in the dependent claims, advantageous further training and improvements of the device specified in the independent claim are possible.

The advantage that can be achieved with the approach presented includes but is not limited to that it provides a practical way to fasten an implant in a vascular canal and remove an implant device from a vascular canal.

An implant device is disclosed herein. In some embodiments, the implant device may include an implant, a fastening device, and a transfer device. The implant may be shaped for implantation into a vascular canal. The fastening device may be connected to the implant (e.g. detachable) and may be movable between a fastening position in which the fastening device may fasten or make the implant fastenable in the vascular canal, and a release position in which the fastening device may release the implant. The transfer device may be coupled to the fastening device and may be adapted to cause transfer of the fastening device between the fastening position and the release position in response to actuation.

In some embodiments, the fastening device may be further configured with a coupling section, a fastening section, and a transfer section. The coupling section may be configured to couple to the implant. The fastening section may be shaped to contact an inside of the vascular canal in an attachment position of the fastening device to secure the implant in the vascular canal. The transfer section may connect the coupling section to the fastening section and may be configured to cause the fastening device to transfer from the attachment position to a release position in response to a push action, in which the fastening device may release the implant.

In some embodiments, a release device may release an implant that is implantable in a vascular canal using the fastening device. The release device may include a sling and an actuating device. The sling may be shaped to wrap around the fastening device in a fastening position in which the fastening device may fasten or make the implant fastenable in the vascular canal. The actuating device may be configured to cause the sling to be tightened when the sling is wrapped around the fastening device to move the fastening device from the fastening position to a release position where the fastening device may release the implant.

The sling may have a flexible loop for wrapping around the fastening device and optionally also additionally a guide rod or guide wire or rigid guide rope or other guide element for guiding the loop.

An actuator for implanting an implant in a vascular canal may include a housing, a slider device and an actuating element. The housing may have a housing opening, an actuating region adjacent to the housing opening, and a movable stop adjacent to the actuating region on an opposite side of the housing opening. The slider device may have a slider section which may be linearly movably received or received in the housing. The actuator may be adapted to cause linear movement of the slider device in an actuating range in response to actuation. The stop may be adapted to prevent linear movement of the slider device when the slider section contacts the stop at a contact position in the actuating range to prevent passage of the slider section out of the actuating range.

The implant may include, for example, a pump configured for use with or as a heart support system. In such an implant, the vascular canal may accordingly be a blood vessel into which the pump may be inserted and shaped to be secured by the fastening device. The pump may be configured to convey a fluid, for example blood, in the vascular canal and for this purpose may, for example, include a rotatable impeller for generating a fluid suction and a drive for driving the impeller. Thanks to the transfer device, the implant presented here can advantageously be quickly and easily transferred to a release position, in which the implant can be gently removed from the vascular canal with just one actuation.

The fastening device may, for example, be a foldable or collapsible frame, the frame being able to be unfolded or deployed or expanded in the fastening position and folded in the release position. In the fastening position, the frame can make radial contact with the vascular canal and, for example, be clamped in the vascular canal. In the release position, the frame cannot contact the vascular canal and can, for example, be arranged adjacent to the implant. In the release position, the implant can be removed from the vascular canal with little or no friction with respect to the vascular canal.

In some embodiments, the fastening device may have a fastening section. In the fastening position, the fastening section of the fastening device may contact an inside of the blood vessel in such a way that the implant is frictionally fastened, for example, clamped in the blood vessel. In some embodiments, the fastening section may contact the inside of the blood vessel radially. In the fastening position, the fastening device may be unfolded, for example clamped. In the fastening position, the transfer section and/or the fastening section may be arranged extending away from the coupling section. In the release position, the fastening device may be folded in, the transfer section and/or the fastening section may be arranged, for example lying against or pressed against the implant. Pressure may be applied to the transfer section, for example, by a release sleeve or a reinforced tube, also known as a retrieval sheath. By continuing pressure actuation, the fastening device may be folded into the release sleeve and the entire implant with the folded fastening device may be accommodated in the release sleeve. The fastening device presented here may thus be advantageously quickly and easily transferred to the release position by simply pressing a button, in which the implant coupled with the fastening device may be gently removed from the blood vessel.

In some embodiments, the fastening device may further be configured to be pulled together by a sling of the release device. A sleeve device of the release device may accommodate the implant and the fastening device surrounding it.

In some embodiments, the fastening device may include the coupling section that may be shaped tubularly to receive an implant in a tubular interior of the coupling section and additionally or alternatively to put a release sleeve over the coupling section. A coupling section diameter may thus be larger than an implant diameter and additionally or alternatively smaller than a release sleeve diameter. Such a tubular coupling section may allow a cylindrical, elongated implant to be accommodated in the interior of the tube. The tubular coupling section and the implant may be mechanically coupled to each other, for example circumferentially. Such a tubular coupling section may also allow the release sleeve to be securely held over or around the coupling section, so that the fastening device may be first pressed into the release position and then fully received together with the implant.

In a mounting position, a coupling section diameter may be smaller than a mounting section span and additionally or alternatively a transfer section span. In this way, the guide sleeve may be received at a narrow point of the fastening device and then the fastening within the vascular canal may be released by pressing on wider sections of the fastening device behind it, which are then, for example, folded in.

In an attached position, the transfer section may also be arranged radially and additionally or alternatively at an angle between the coupling section and the fastening section. Radial shaping of the transfer section may allow the guide sleeve to be fully folded and receive the internal implant with the fastening device. A tapered fastening section may allow the guide sleeve to slide easily along the transfer section with little force when the fastening device is transferred to the release position.

It may also advantageous if at least one section of the fastening device has a shape memory and additionally or alternatively self-expanding material according to a design form. These sections may be, for example, the transfer section and additionally or alternatively the fastening section. The material may be a nickel-titanium alloy, for example Nitinol. Such a material may enable the fastening device to be transferred independently to the fastening position.

According to an example, the fastening section may have at least three fixing arms shaped to make radial contact with a duct at three contact points in the fastening position. This may create both a gentle and safe way of securing the implant in the vascular canal.

In some embodiments, the fastening device arms may be configured as a holding frame. The holding frame for an implant device may include at least two feet for placing the implant in a vascular canal and at least one X-ray marker adapted to enable a position of at least one of the feet in the vascular canal to be checked using X-ray light. The vascular canal, in some instances, may be an aorta.

The holding frame may be a lattice structure, which may include Nitinol and may be configured to hold an implant, such as a cardiac support system, securely and to be placed inside an aorta or implanted between three valve leaflets of the aorta. The holding frame may include at least two feet. In some embodiments, one foot may be a clamp or a support element that may be used to hold the holding frame or the cardiac support system in a certain, desired position in the aorta.

A cardiac support system may be a heart pump, for example which may be implanted inside the aorta by means of at least one holding frame and held there. An X-ray marker may be a structure that is clearly distinguishable from another structure in X-rays and may thus be used by a surgeon to help verify a position of the cardiac support system during surgery and during follow-up. This may be done with the use of X-rays, as these make the X-ray marker visible.

Short-term intravascular cardiac support systems are delivered or implanted either percutaneously (through the skin) or surgically via various arterial accesses (e.g. femoral artery). The final positioning of the systems is visually supported intraoperatively (e.g. via ultrasound or fluoroscopy). However, these systems can have a high risk of dislocation due to the lack of local fixation. Current Nitinol frames do not provide the surgeon with any indication of their rotational alignment in a longitudinal axis. A novel VAD system or a cardiac support system, which is to be placed minimally invasively within the aorta, can enable the precision required for implantation during alignment.

The VAD system presented here, and in particular the pump, may be supported by a Nitinol frame, for example, which may also be used as a holding frame that is placed and anchored within the aorta. To ensure good function of the cardiac support system, the holding frame may be positioned with a front surface parallel and centric to the aortic valve. The Nitinol frame may include three feet for stable anchoring. During implantation, these may be positioned to match the aortic valve and may be placed between the three leaflets.

With previous VAD systems, the two-dimensional nature of an X-ray image meant that the surgeon had little information to assess the rotational alignment in the longitudinal axis. The approach presented here makes it possible, through additional indicators in the holding frame, which is shown here as a Nitinol frame, to perform this positioning process quickly and safely. This results in reduced operating time, avoidance of incorrect positioning and thus risks for the patient. The positioning can also be easily checked in a follow-up check and any tilting can be quickly detected.

The X-ray marker may be arranged according to a design on at least one foot of the holding frame, in particular where the X-ray marker has a structure different from that in an X-ray image of another component of the holding frame. This offers the advantage that during the operation as well as during the follow-up, with the help of X-rays, the X-ray markers can be easily and reliably detected and thus the positioning of the holding frame can be checked. The X-ray marker can provide clues that can enable a surgeon to verify the exact alignment of the holding frame and thus of the cardiac support system.

The holding frame and/or the feet may be configured as a grid structure according to an example. The grid structure may be a structure with which a wire may be bent into a grid to form a foot and/or to form an X-ray marker. Such a design offers the advantage of being able to realize the X-ray marker with technically simple and cost-effective means.

According to some embodiments, the X-ray marker may have a different fill structure than another area of the holding frame. A fill structure may be a structure that fills at least part of a bounded area of the foot or holding frame. This may create a visual contrast that makes it easy for a surgeon or physician to identify the position of the holding frame.

According to an example, the X-ray marker may form a symbol that may be distinguished from another area of the holding frame. A symbol may be a predetermined structure of the X-ray marker which is clear and easy to recognize. This also creates a visual contrast that allows for a secure and unambiguous identification of the positioning of the holding frame.

In addition, the X-ray marker may be configured such that it can distinguish a further area of the holding frame having a distinctive design or shape of an outer contour. This also creates a visual contrast that allows the positioning of the holding frame to be clearly identified.

According to an example, the X-ray marker may have a size that may be distinguishable from another area of the holding frame. This also creates a visual contrast that allows the positioning of the holding frame to be clearly identified.

The X-ray marker may have a pattern that may be distinguishable from a further area of the holding frame according to an example. This also creates a visual contrast that allows clear identification of the position of the holding frame.

According to an example, the at least two feet of the holding frame may position the holding frame between valve leaflets of an aortic valve. This may help improve function of the cardiac support system. The approach presented here may reduce hazards to the patient, such as a malfunction of the cardiac support system or discontinuation of therapy with the cardiac support system if the device is incorrectly positioned. Thus, a risk of slipping may be avoided or at least reduced by anchoring the device at an implantation site using the holding frame.

According to an example, the holding frame may at least partially include a Nitinol material. Nitinol combines the advantages of biocompatibility and superelastic properties, which may allow even complex structures to be delivered and compressed in a small space.

According to an example, the holding frame may be concentrically or otherwise coupled or connected to the cardiac support system. The holding frame may be permanently, semi-permanently, or temporarily coupled to the cardiac support system. The coupling can help ensure a reliable support of the cardiac support system in the aorta.

In some embodiments, the transfer device may include at least one pull cord, which may be shaped to cause, in response to actuation in the form of a pulling movement, transfer of the fastening device from a fastening position to a release position and/or, in response to further actuation in the form of a release movement, transfer of the fastening device from a release position to a fastening position. The pull cord may also be attached to the fastening device by at least one cable to pull the fastening device towards the implant in response to a traction movement. The fastening device may be pretensioned to the implant so that a release movement may be affected in response to release or slackening of the pull cord.

In some embodiments, the pull cord may include a pull cord driver configured for coupling with an operating device for operating the transfer device. The pull cord driver may be located at an end of the pull cord opposite wires. Such a pull cord driver is advantageous in that it may enable the transfer device to be operated automatically, for example to cause actuation.

In some embodiments, the transfer device may be configured as a release device. The release device may include a control that may be shaped to provide, in response to a rotary action and, in addition or alternatively, a sliding action and, in addition or alternatively, a pushing action, tightening of the sling. The loop of the sling around the fastening device, for example the frame, may be tightened in response to the control(s).

The control may further be adapted to cause the loop to open and, additionally or alternatively, extend the loop and, additionally or alternatively, wrap the fastening device with the loop in response to a rotary action, and additionally or alternatively, a sliding action and, additionally or alternatively, a pushing action. The opening/extending/wrapping rotary control/sliding control/pressure control may be before the tightening rotary control/sliding control/pressure control. The opening/extending/wrapping rotary control may be in an opposite direction to a tightening rotary control. The opening/extending/wrapping sliding action may be in an opposite direction to a tightening sliding action.

In some embodiments, the releasing device may include an actuating member adapted to accommodate some or all of an opening/extending/wrapping rotary actuation, an opening/extending/wrapping sliding actuation, an opening/extending/wrapping pushing actuation, a tightening rotary actuation, a tightening sliding actuation, and/or a tightening pushing actuation. In this way, the actuations may be carried out by a single actuating member or different actuating members.

The release device may further include a sleeve device shaped to receive the fastening device in the release position and/or the implant. The sleeve device may accommodate the above-mentioned elements in a protective manner, which may enable gentle removal of the implant.

The sleeve device may also include a sliding device which may be located within the sleeve device and may be additionally or alternatively configured to resist a force in a longitudinal direction of the sleeve device. This provides stability in the application of the release device.

According to an example, the actuating member may be configured to cause, in response to a further rotary actuation and, additionally or alternatively, further sliding actuation and, additionally or alternatively, further pressure actuation, an extension of the sleeve device over the fastening device in the release position and, additionally or alternatively, the implant. The further rotary actuation may be affected transversely to the opening/extending/wrapping rotary actuation and, additionally or alternatively, tightening rotary actuation. The further sliding actuation may be performed transversely to the opening/extending/wrapping sliding actuation and, additionally or alternatively tightening sliding actuation. The control may have a further actuating member configured to accommodate the further rotary actuation and, additionally or alternatively, further sliding actuation and, additionally or alternatively, further pushing actuation. When the sleeve device is extended, a linear movement of the sleeve device towards the implant may be affected until the implant is accommodated in the sleeve device together with the fastening device.

The release device may enable the implant to be quickly and easily transferred into the release position, in which the implant may be gently removed from the blood vessel. The release device may be advantageously used for any implant with a fastening device. The release device may also be used to remove the implant from the blood vessel.

In some embodiments, the implant device may include an actuator configured for use in conjunction with a delivery system, which may include the actuator and the implant to be implanted. The actuator may be configured to implant the implant in the vascular canal, for example by actuation or various actuations of the actuator to transport the implant coupled to the actuator to a desired position in the vascular canal and, additionally or alternatively, to release or fix it in that position. The actuator may be intended for manual operation and may be advantageously operated with one hand. A first runner section may be an end section of a runner device. A further runner section opposite the first runner section may be led out of the housing opening for coupling to the implant or may be arranged to be led out. The actuator may be mechanically coupled to armature, for example the actuator may be rotatably formed so that a linear movement of the armature may be affected in response to actuation of the actuator in the form of a rotary actuation. The actuator may, for example, be a rotary knob, where the actuation may be, for example, a manual rotary actuation on the rotary knob. An additional slider section of the slider device adjacent to the slider section may be arranged, for example, out of the housing opening and, additionally or alternatively guided through the rotary knob. Such an actuating device makes it possible to operate at least one functional area of the actuating device, for example to move the implant coupled to the actuating device, by one-handed actuation.

The housing may include a second setting range, where a stop device may be located between a setting range and the second setting range. Within the second setting range, a second functional range of the actuating device may be operated, for example moving or releasing or decoupling the implant from the actuating device. Passing of the slider device into the second setting range may be prevented by the stop device. This prevents unintentional passage into the second setting range, in which another function of the actuating device may be performed.

According to an example, the release device may be configured to allow the actuating device to include at least one release unit adapted to open the stop in response to a release movement when the slider section is positioned in the contact position to allow passage of the slider to a second range of travel. The release device for the actuator thus may enable, by making a conscious decision which may be signaled by the release movement, a transition from a functional area assigned to an actuating area to a second actuating area in which another functional area of the actuating device may be operated.

For use with the actuator, the release device may be actuated by means of a control element. The release device can be configured here, for example, to open the stop device in response to the release movement, which is configured as an actuating element linear movement of the actuating element, in particular a pulling out of the actuating element, when the slider section is arranged in the contact position. “Pulling out” can be understood as pulling the actuating element away from the housing. This allows the actuating device to be operated in two different setting ranges, which can perform different functions, by operating only the one actuating element. All mentioned actuations and movements on the actuating element can be carried out with one hand.

The release device may further be adapted to close the stop device in response to a locking movement when the slider section is in the contact position, to prevent the slider section from passing into the second setting range. In other words, the stop can be kept closed by the locking movement. This also allows a conscious decision not to operate the second functional range to be made.

According to one design form, the blocking movement of the release device can represent a movement opposite to the release movement. Thus, the locking movement can be an actuating element linear movement of the actuating element opposite to the actuating element linear movement, for example a pushing movement of the actuating element towards or against the housing. In this way, many simple actuations and movements of the actuating element can be realized to operate different functions.

It is further advantageous if the release device is also configured to close the stop device as soon as the slider device is positioned in the second setting range, in order to prevent the slider section from passing back into the setting range. In this way an obviously separate controllability can be achieved, whereby a return to a closed functional area, for example, is prevented.

According to one design form, the actuating element can be configured to affect a linear return movement of the slider device in the actuating range in response to an actuation opposite to the actuation. Accordingly, the actuating element can be configured to affect the opposite linear return movement of the 941 device in the second setting range in response to the opposite actuation. Thus, linear movements within the setting range and additionally or alternatively within the second setting range in two directions are possible, for example to carry out an adjustment of a movement.

In alternative embodiments, the actuator can be configured with a guide device and a catheter system. The catheter system is coupled or couplable to the guide device and comprises a release device for implanting the implant and a bendable movement device, movable with respect to at least part of the guide device, for moving the release device. The guiding device is configured to control (for example mechanically) a bending operation of the moving device. With the actuating device presented here and in particular the guide device of this actuating device, an insertion process of the implant by means of the release device moved by the moving device is possible in a particularly gentle manner, since the guide device is configured to bend the moving device, for example, along a complexly curved channel. Thus, during implantation, pressure on vascular canal walls of the vascular canal in vascular canal bends can be minimized.

The guide device may, for example, comprise a flexible outer guide unit in or on which an inner portion of the moving device is received or receivable to bend the inner portion in response to a bending movement of the outer guide unit caused by the guide device. In this way, the moving device accommodated on or in the guide device can be moved along, for example within a flexible tubular guide device. Such an external guide unit enables both a circumferential bending of the movement device and protection for the movement device.

However, the guide device may also include a flexible inner guide unit which is received or receivable in a section of the movement device to bend the section in response to a bending movement of the inner guide unit caused by the guide device. In such a configuration, the portion of the moving device may be flexible and tubular to accommodate the guide device. A particularly slim system of movement device and guiding device is possible.

The movement device can be linearly movable and additionally or alternatively rotationally capable. A linear movement or rotation of the motion device may, for example, be in response to one or more operations of a catheter system control element provided for this purpose. The linear movement can be used to insert the release device with the implant into the channel. The rotation can support the insertion or release the implant.

It is also advantageous if the movement device is coupled or can be coupled rotationally and additionally or alternatively translationally free with respect to at least one guide device section of the guide device. In this way, the moving device and the guide device can be coupled with each other without influencing each other by mechanical movements.

The catheter system and, in addition or alternatively, the guide device may be configured to block movement of the motion device. The mobility may be blocked in response to a lock signal or actuator lock actuation, for example. This provides a means of deliberately setting a function of the control device and avoiding unintentional actuation.

The catheter system and additionally or alternatively the guiding device may also be configured to enable or cause linear motion and additionally or alternatively rotational motion of the motion device. The linear motion and additionally or alternatively rotational motion may be enabled or caused, for example, in response to an enabling signal or an enabling actuation of the control device. This creates a possibility for consciously setting a further function of the actuating device.

The catheter system and additionally or alternatively the guiding device may be configured to block linear motion of the motion device and additionally or alternatively to release or cause rotational motion of the motion device. The linear motion of the device may be disabled in response to a control signal or control actuation. Additionally or alternatively, the rotational mobility of the movement device may be enabled or caused in response to the control signal or control actuation or enabled or caused in response to a further control signal or control actuation. This provides a means of deliberately setting an additional function of the control device.

The implant device disclosed can feature a combination of the above aspects. For example, in some embodiments, the implant device may be configured to have a fastening device that has a fastening position and is foldable and also have an actuator with a guiding system and catheter. The fastening device works to clamp the implant system into place in the vascular canal and the actuator can extend from the fastening device navigate around bends in the vascular canal to release the implant at the desired location.

In some embodiments that example may further include the holding frame that will show through the use of the X-ray process, that the fastening device is clamped at the proper location for implantation. The actuator can extend from there and the user will know the length and orientation of the catheter in order to deliver the implant to the exact spot the user wants the implant to go.

A separate example of the implant device may include a release device controlling the holding frame. The holding frame can clamp and indicate that is clamped in the proper location and then can be removed with the loop.

It should be appreciated by those knowledgeable in the art that many additional combinations of the functional pieces of the implant device can be utilized to create the optimal configuration for the circumstances. These circumstances can include the type of implant that is being inserted and the location inside the vascular system of the implant. The vascular system is complex and implementation of an implant in one section of the vascular system may require the benefits of a catheter while another may not. Configurations that include the catheter may not be desirable when implanting into a section of the vascular system that is easily accessible without bending through a vascular canal such as a blood vessel.

In some embodiments the implant device further includes an operating device that couples to the transfer device and, in response to an operation, actuates the transfer device. Such an operating device can be used to actuate the transfer device manually or automatically.

According to one design, the operating device may have a slider device which is configured to be coupled with a pull cord driver of a pull cord of the transfer device. This allows a mechanical coupling between the operator interface and the transfer device.

The operator control may also be configured to operate in a coupled condition with the transfer device in response to a rotary control.

It is further advantageous if the operating device, according to a design, has a supporting extrusion device for receiving at least a portion of the transfer device in order to resist a compressive force during operation or actuation. In this case, the supporting extrusion device can, for example, accommodate a section of the pull cord in order to resist the compressive force of the pull cord during operation.

An implant system may have one of the previously presented implant devices and one of the previously presented operating devices. Such an implant system realizes a fully slender system for safe removal of the implant device from a vascular canal. In some embodiments, the removal is carried out by placing a guide sleeve over the implant system.

In some embodiments the implant system may include a sensor cable or shaft carrying electrical conductors that may be used to electrically communicate between the implant 305 and an external console that may provide power to the implant or detect sensor signals from the implant or other functions. The sensor cable may be configured to guide the transfer device. The transfer device, further configured as the release device can include the sensor cable which is passed through a loop of the sling and/or which runs along a guide rod of the sling. The sensor cable may be configured as one of the pull cords of the transfer device. This enables the release device to be guided safely to the implant and additionally or alternatively the loop around the fastening device.

A method for manufacturing one of the previously presented implant devices is also presented. The method includes a step of providing and a step of coupling. The providing step provides an implant for implantation in a vascular canal and a fastening device connected (for example, releasably) to the implant, which is movable between a fastening position in which the fastening device fastens or makes fastenable the implant in the vascular canal and a release position in which the fastening device releases the implant. In the step of coupling, a transfer device is coupled to the fastening device, the transfer device being adapted to cause transfer of the fastening device between the fastening position and the release position in response to actuation.

A method for manufacturing an actuating device is also presented. The method comprises a step of providing and a step of assembling. The providing step provides a housing having a housing opening, an actuating portion adjacent to the housing opening and a movable stop adjacent to the actuating portion on an opposite side of the housing opening, a slider means and an actuator. In the assembling step, the housing, the slider means and the actuator are assembled such that a slider portion of the slider means is linearly movably received in the housing, and that the actuator is arranged to cause linear movement of the slider means in the actuating range in response to actuation, the stop is arranged to prevent the linear movement of the slider when the slider portion contacts the stop at a contact position in the actuating range to prevent the slider portion from passing out of the actuating range.

A method for manufacturing the actuator configured with the guiding device and catheter system is also presented. The method comprises a step of providing and a step of coupling. The provision step provides a guide device and a catheter system with a release device for implanting the implant and a bendable movement device for moving the release device. In the coupling step, the guide device and the catheter system are coupled such that the moving device is movable with respect to the guide device and that the guide device is arranged to mechanically control a bending operation of the moving device to produce the actuating device.

This procedure can be implemented, for example, in software or hardware or in a mixture of software and hardware, for example, in a control unit.

The approach presented here also creates a device that is configured to carry out, control or implement the steps of a variant of a process presented here in appropriate facilities. This design variant of the approach in the form of a device can also be used to solve the task underlying the approach quickly and efficiently.

For this purpose, the device may have at least one computing unit for processing signals or data, at least one memory unit for storing signals or data, at least one interface to a sensor or an actuator for reading in sensor signals from the sensor or for outputting data or control signals to the actuator and/or at least one communication interface for reading in or outputting data embedded in a communication protocol. The computing unit may be, for example, a signal processor, a microcontroller, or the like, wherein the memory unit may be a flash memory, an EEPROM or a magnetic memory unit. The communication interface may be configured to read or output data wirelessly and/or by wire, whereby a communication interface which can read or output wire-bound data can read or output these data, for example electrically or optically, from a corresponding data transmission line or output them into a corresponding data transmission line.

A device in this case can be understood as an electrical device that processes sensor signals and outputs control and/or data signals as a function thereof. The device may have an interface, which may be hardware and/or software-based. In the case of a hardware configuration, the interfaces can, for example, be part of a so-called system ASIC, which contains various functions of the device. However, it is also possible that the interfaces are separate integrated circuits or at least partly consist of discrete components. In the case of a software-based education, the interfaces can be software modules, which for example exist on a microcontroller next to other software modules.

A computer program product or computer program with program code, which may be stored on a machine-readable carrier or storage medium, such as a semiconductor memory, a hard disk memory or an optical memory, and which is used to carry out, implement and/or control the steps of the process according to one of the forms of execution described above, is also advantageous, in particular if the program product or program is executed on a computer or device.

### BRIEF DESCRIPTION OF THE DRAWINGS

Execution examples of the approach presented here are shown in the drawings and explained in more detail in the following description. It shows:

FIG. 1 illustrates an implant device shown implanting an implant comprising a heart pump inside a heart.

FIG. 2 illustrates an implant device endovascularly inserted into the vascular system via a patient's leg to implant an implant into the heart.

FIG. 3 illustrates a schematic side view of an implant device having a heart pump according to a design example.

FIG. 4 illustrates a schematic side view of an implant system with an implant device and an operating device for operating the implant device according to an execution example.

FIG. 5 illustrates a schematic side view of an implant system arranged in the release position according to a design example.

FIG. 6 illustrates a schematic side view of an implant system with an implant and a release device for releasing the implant according to a design example.

FIG. 7 illustrates a schematic side view of an implant system prior to the rotary actuation of the control according to a design example.

FIG. 8 illustrates a schematic side view of an implant system sling tightening and the frame folding over the sleeve device according to a design example.

FIG. 9 illustrates schematic side view of an implant system with an implant and a fastening device for fastening the implant in a channel according to a design example.

FIG. 10 illustrates a schematic cross-sectional view of a fastening device according to a design example.

FIG. 11 illustrates a schematic side view of an implant system arranged in the release position according to a design example.

FIG. 12 illustrates a schematic cross-sectional view of a fastening device in the release position according to an example of design.

FIG. 13 illustrates a schematic side view of an actuator for implanting an implant in a vascular canal according to a design example.

FIG. 14 illustrates a schematic side view of an actuator for implanting an implant in a vascular canal after performing linear movement of the slider.

FIG. 15 illustrates a schematic side view of an actuator for implanting an implant in a vascular canal, the slider section of the actuator arranged in the contact position.

FIG. 16 illustrates a schematic side view of an actuator for implanting an implant in a vascular canal, the slider device of the actuator positioned in the second setting range.

FIG. 17 illustrates a schematic side view of an actuator with a guiding device and a catheter system for implanting an implant in a vascular canal.

FIG. 18 illustrates a schematic side view of an actuator for implanting an implant in a vascular canal with a bendable outer guide unit.

FIG. 19a illustrates a schematic side view of the actuating devices of an actuator with blocked mobility.

FIG. 19b illustrates a schematic side view of the actuating devices of an actuator that are moveable.

FIG. 20 illustrates a schematic side view of the actuating devices of an actuator that are prevented from moving linearly.

FIG. 21 a schematic representation of an example of a holding frame for a cardiac support system.

FIG. 22 in four partial figures each a schematic representation of a Design examples of an X-ray marker of a holding frame for a heart support system.

FIG. 23 a flow chart of a process for manufacturing an implant device according to an execution example.

### DETAILED DESCRIPTION

In the following description of favorable execution examples of the present approach, the same or similar reference signs are used for the elements represented in the different figures and which have a similar effect, whereby a repeated description of these elements is omitted.

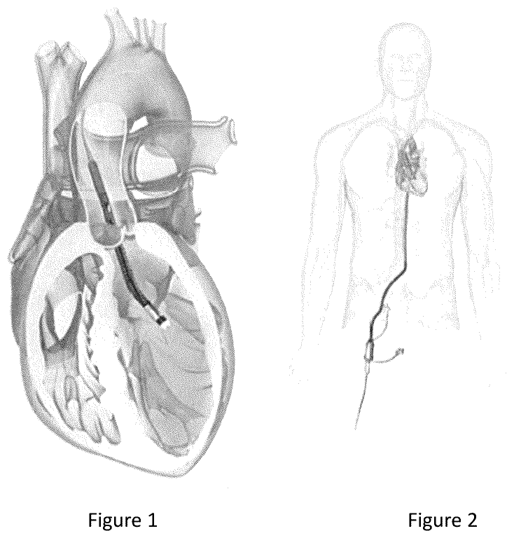

FIG. 1 shows a rendering of the implant device. The implant device of FIG. 1 delivers enters into the heart and deploys the implant at the proper place within the heart space. The implant device can be configured and adapted in a variety of ways, herein disclosed, depending on the location the implant device is to be implanted.

FIG. 2 similarly shows the insertion of the implant device system through a vein in the leg to reach the aorta of the heart. Once positioned in the heart, the implant device deploys an implant into the cardiovascular system.

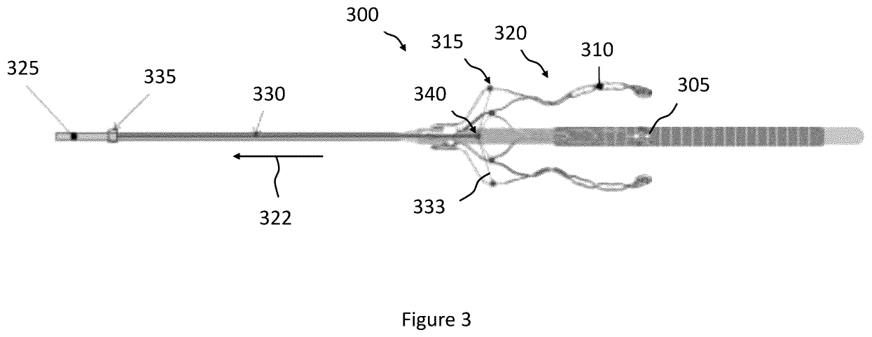

FIG. 3 shows a schematic side view of an implant device 300 according to a design example.

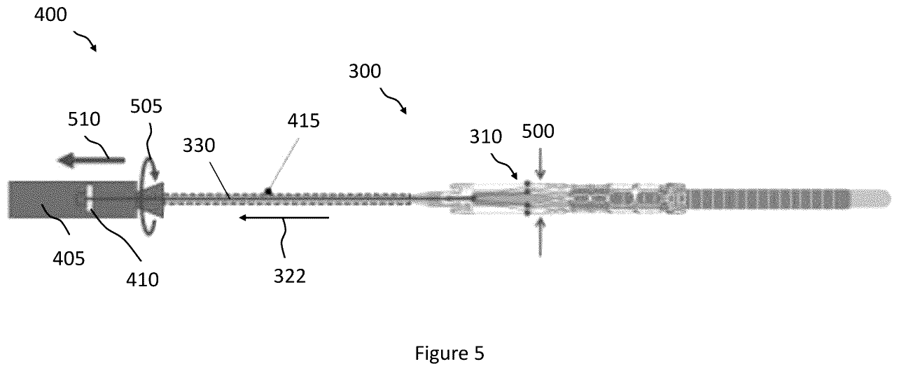

The implant device 300 has an implant 305, a fastening device 310 and a transfer device 315. The implant is shaped for implantation into a vascular canal. The fastening device 310 is connected to the implant 305 (e.g. detachable) and is movable between a fastening position 320, in which the fastening device 310 fastens or makes fastenable the implant 305 in an anatomical space such as the vascular canal, and a release position, in which the fastening device 310 releases the implant 305. The transfer device 315 is coupled to the fastening device 310 and is adapted to cause transfer of the fastening device 310 between the attachment position 320 and the release position in response to an actuation 322. The release position is shown in FIG. 5.

According to this design example, implant 305 is only an example of a pump that is configured for use for or as a heart support system. According to this example, the implant 305 is configured to be implanted in a blood vessel and can be fixed there in the fastening position 320 shown here by means of the fastening device 310. The blood vessel may be the aorta, for example. The pump is formed according to this design example for conveying a fluid, in this case blood, in the vascular canal and for this purpose has, according to this design example, a rotatable impeller for generating a fluid suction and a drive device for driving the impeller and further may have at least one sensor and a sensor cable 325. According to an alternative design example, the implant 305 is configured as any other object or device that can be implanted in an anatomical space such as a vascular canal or as another minimally invasive implantable device.

According to this design example, the fastening device 310 is configured as a collapsible (e.g., foldable) frame, whereby the frame is deployed (e.g., unfolded) in the fastening position 320 and collapsed (e.g., folded) in the release position. In the fastening position 320 the frame is arranged to contact the duct radially and to be clamped in the duct according to a design example. In the release position the frame does not contact the vascular canal according to an example and is arranged adjacent to implant 305 according to an example.

According to this design example, the transfer device 315 has at least one pull cord 330 which is formed for the purpose of affecting, in response to actuation 322 in the form of a pulling movement, the transfer of the fastening device 310 from the fastening position 320 into the release position and/or, in response to a further actuation in the form of a release movement, the transfer of the fastening device 310 from the release position into the fastening position 320. According to this design example, the pull cord 330 is attached to the fastening device 310 by means of a cable 333 or several cables 333 in order to pull the fastening device 310 towards the implant 305 in response to the traction movement. The fastening device 310 is pretensioned according to an example so that the release movement can be affected in response to a release or yielding of the pull cord 330. The pull cord 330 is also arranged according to this example on or in the sensor cable 325 of the implant 305 or guided around the sensor cable 325.

Furthermore, according to this design example, the pull cord 330 has a pull cord driver 335 and/or a cable junction 340 of the cables 333. According to this design example, the pull cord driver 335 and the cable junction point 340 are arranged at opposite ends of the pull cord 330. The pulling cable driver 335 is configured for coupling with an operating device for operating the transfer device 315. According to this design example, at least four cables 333 attached to four frame sections of the frame extend radially from the cable junction point 340 away from the cable junction point 340. According to an alternative design example, more or less than four cables 333 attached to frame sections of the frame extend radially from the cable junction 340.

The implant device 300 presented here enables a so-called retrieval procedure or the removal of an implant 305 with the aid of an integrated pull cord 330, which enables the removal of the implant 305, which is anchored in a vascular system with a collapsible fastening device 310. Connecting points, here in the form of the pull cord driver 335 and/or the sensor cable 325, for the retrieval system remain accessible during the implantation of the implant 305 on the patient according to an execution example. The implant device 300 has the fastening device 310 in the form of the collapsible frame, which can be actuated and removed by means of a pulling mechanism and, according to a design example, by means of a support extrusion outside the body.

FIG. 3 shows how an access to the sensor cable 325 and the pull cord driver 335 is created.

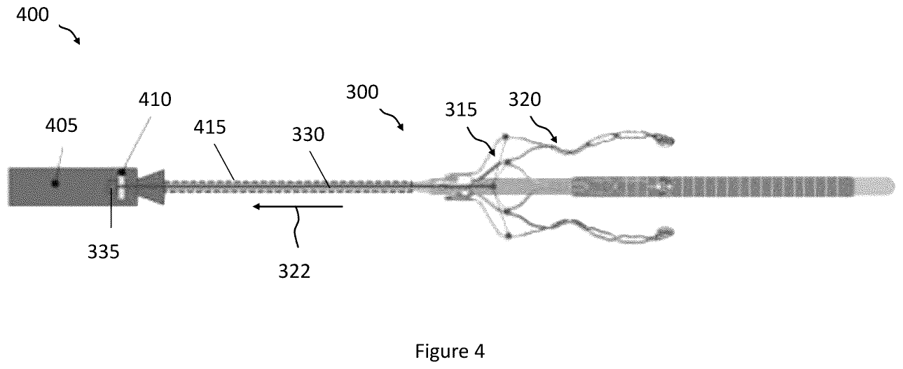

FIG. 4 shows a schematic side view of an implant system 400 with an implant device 300 and an operating device 405 for operating an implant device 300 according to a design example. This can be the implant device 300 described in FIG. 3.

The implant system 400 features the implant device 300 and also the operating device 405. The operating device 405 is configured for operating the transfer device 315 of the implant device 300, wherein the operating device 405 is coupled to the transfer device 315 for this purpose according to this design example and is configured to cause actuation of the transfer device 315 in response to an operation, for example collapse in response to increased tension 322, or expansion in response to released tension.

According to this design example, the operating device 405 has a slider unit 410 which is coupled to the pull cord carrier 335 of the pull cord 330 of the transfer device 315. The operator control device 405 may be further configured in accordance with this design example to cause actuation 322 in a coupled state with the transfer device 315 in response to an operation configured as a rotary movement.

According to this design example, the operating device also has a supporting extrusion device 415 for receiving at least a section of the transfer device 315 in order to resist a compressive force during operation and/or actuation. According to this design example, a section of the pull cord 330 is accommodated by the supporting extrusion device 415 in order to resist the compressive force of the pull cord 330 during operation.

The support extrusion connected to the “retrieval handle” in the form of the operating device 405 can be inserted into the vessel system via the sensor cable or shaft. The sensor cable or shaft, for example, which is already in the body, is used as a guide for the catheter system according to a design example. The Implant System 400 now allows a minimally invasive procedure that can be performed quickly and places little stress on the patient.

A so-called retrieval sheath, which could be realized in the form of a sleeve, for example, which could be slipped over the implant 305 and/or the fastening device 320, is not necessary in this design example, whereby an overall system of the implant system 400 remains advantageously very slim.

FIG. 5 shows a schematic side view of an implant system 400 according to a design example. This may be the implant system 400 described in FIG. 2, with the difference that the fastening device 310 is arranged in the release position 500, in which the frame is positioned adjacent to the implant 305.

According to this design example, the release position 500 was brought about by means of a rotary movement 505 of an actuator of the operating device 405 coupled to the pull cord 330. By means of the rotary motion 505, the slider unit 410 was moved linearly into an interior of the operating device 405, thus pulling the coupled pull cord 330 into the operating device 405. By continuing or holding steady the rotary movement 505 in addition to a linear movement 510 of the operating device 405, the implant 305 can be pulled out of the vessel in a linear fashion according to a design example.

Here it is shown how the pull cord 330 is pulled in according to a design example with the aid of the operating device 405, which can also be called a “handle”, whereby the support extrusion device 415 resists the compressive force.

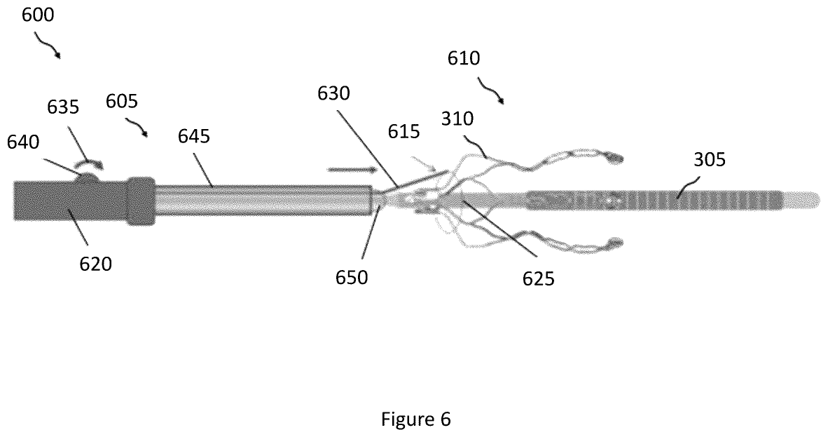

FIG. 6 shows a schematic side view of an implant system 600 with an implant 305 and a fastening device 310 for anchoring the implant 305 when deployed and releasing the implant 305 when collapsed according to a design example.

The implant 305 may be shaped to be implanted in a vascular canal and has a transfer device 615 for fastening the implant 305 in the vascular canal. According to this design example, the transfer device 615 is arranged in a fastening position 610, in which the fastening device 615 fastens or makes it possible to fasten the implant 305 in the vascular canal.

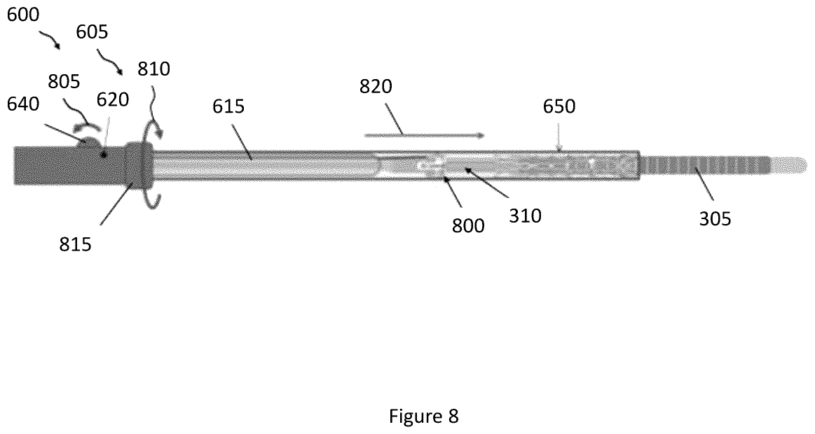

The release device 605 has a sling 615 and an operating device 620. The sling 615 is configured to wrap around the fastening device 310 in the fastening position 610. The actuating device 620 is configured to cause the sling 615 to be tightened when the sling 625 is wrapped around the fastening device 310 in order to move the fastening device 310 from the fastening position 610 to a release position in which the fastening device releases the implant 305. In FIG. 6 the fastening device 310 is shown in a state where the sling 615 is already wrapped around the fastening device 310, but before tightening the sling 625. The release position is shown in FIG. 8.

According to this design example, the sling 625 has a flexible loop 625 which wraps around the fastening device 310 according to this design example and/or a guide rod 630 or, according to an alternative design example, a guide wire or rigid guide rope which guides the loop 625 and/or mechanically connects it to the control device 620.

According to this design example, implant 305 is a pump configured for use with or as a cardiac support system, for example. According to this design example, the implant 305 is configured to be implanted in a blood vessel and can be fixed there in the fastening position 610 shown here by means of the fixing device 310. The blood vessel may be the aorta, for example. The pump is formed according to this design example for conveying a fluid, in this case blood, in a channel and for this purpose has, according to this design example, a rotatable impeller for generating a fluid suction and a drive device for driving the impeller and further may have at least one sensor and a sensor cable. According to an alternative design example, the implant 305 is configured as any other object or device which can be implanted into a channel or as another minimally invasive implantable device.

According to this design example, the fastening device 310 is configured as a foldable frame, whereby the frame is unfolded in the fastening position 610 and folded in the release position. In the fastening position 610 the frame is arranged to contact the duct radially and to be clamped in the duct according to a design example. According to this design example, the frame is pretensioned in such a way that it automatically moves into the fastening position 610. In the release position, the frame does not contact the channel according to a design example and is arranged adjacent to the implant 305, pulled together by the loop 625 according to a design example.

The operating device 620 is shaped in accordance with this design example in order to cause the sling 625 including flexible loop 625, to be tightened in response to a rotary actuation and/or sliding actuation and/or pressure actuation. This rotary actuation 810 is shown in FIG. 8. The operating device 620 is further configured, according to this example, to cause opening and/or extending of the sling 625 and/or wrapping of the fastening device 310 with the sling 625 in response to a preceding rotary actuation 635 and/or, according to an alternative example, preceding sliding actuation and/or preceding pushing actuation. According to this design example, the rotary actuation 635 preceding the rotary actuation and/or sliding actuation and/or pressure actuation has been affected, whereby the sling 625 has been opened, extended and placed over the implant 305.

According to this design example, the release device 605 comprises an actuating element 640 configured to receive the rotary actuation and/or sliding actuation and/or push actuation and/or preceding rotary actuation 635 and/or preceding sliding actuation and/or preceding push actuation.

According to this design example, the release device 605 also has a sleeve device 645, which is configured to receive the fastening device 310 in the release position and/or the implant 305. According to this design example, the sleeve device 645 also has a pusher device 650, also referred to as pusher, which is arranged or arrangeable in the sleeve device 645 and/or is configured to resist a force in the longitudinal direction of the sleeve device 645.

The release device 605 presented here enables the removal of an implant 305, which is anchored in a vascular system with a collapsible frame, by means of a loop 625. The release device 605 advantageously realizes a universally applicable retrieval system, which establishes a connection/force transmission to the implant 305 by means of the loop 625. With the release device 605 a variety of different implants 305 in different implant sizes can be operated, since the loop 625 may be universally applicable.

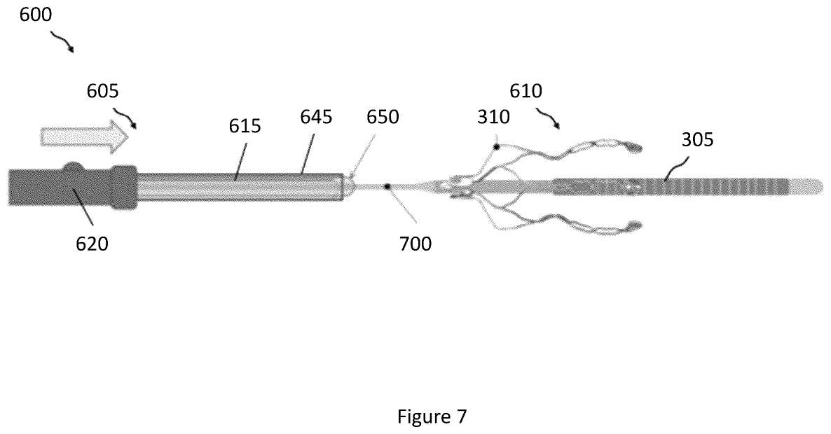

FIG. 7 shows a schematic side view of an implant system 600 according to a design example. This can be the implant system 600 described in FIG. 6.

According to this design example, an initial position of the implant system 600 is shown before the preceding rotary actuation of the operating device 620 described in FIG. 6.

The release device 605, which can also be described as a “retrieval system”, may be inserted to implant 305 according to this design example via the sensor cable 700 of implant 305. According to this example, the sensor cable 700 is guided through the loop 625 of the sling 615 and/or runs along beside the guide rod 630 of the sling 615. The entire loop 615 is accommodated in the sleeve device 645 according to this example.

FIG. 8 shows a schematic side view of an implant system 600 according to a design example. This can be the implant system 600 described in FIG. 6 or 7, which is arranged in the release position 800.

According to this design example, the fastening device 310, which is configured as a foldable frame, is folded in the release position 800 and/or arranged adjacent to the implant 305. According to this design example, the release position 800 was affected in response to the rotary actuation 805 of the actuator 640, whereby the rotary actuation 805 according to this design example was affected in an opposite direction to the preceding rotary actuation shown in FIG. 6. According to an alternative design example, the operating device 620 comprises different actuators 640 for performing one or more of the actuations 805 or one or more of the preceding actuations.

In accordance with this design example, the operating device 620 is also configured to cause the sleeve device 645 to be extended via the fastening device 310 in the release position 800 and/or the implant 305 in response to a further rotary actuation 810 and/or further sliding actuation and/or further pressure actuation. The further rotary actuation 810 was caused according to this design example transversely to the rotary actuation 805 and/or preceding rotary actuation. For this purpose the operating device 620 according to this design example has a further actuating element 815 which is configured to accommodate the further rotary actuation 810 or according to an alternative design example the further sliding actuation and/or further push actuation. In response to the further rotary actuation 810, a linear movement 820 of the sleeve device 645 was caused on the implant 305 in accordance with this design example until the implant 305 was accommodated in the sleeve device 645 together with the fastening device 310 in the release position 800. One position of the actuating device 620, on the other hand, is unchanged according to this design example.

In summary, FIG. 8 shows how the sling 625 was tightened and then the frame 310 was folded over the sleeve device 645, which can also be described as Retrieval Sheath.

The implant 305 can now be removed from the vascular canal according to a design example by means of the release device 605, e.g. it can be pulled out.

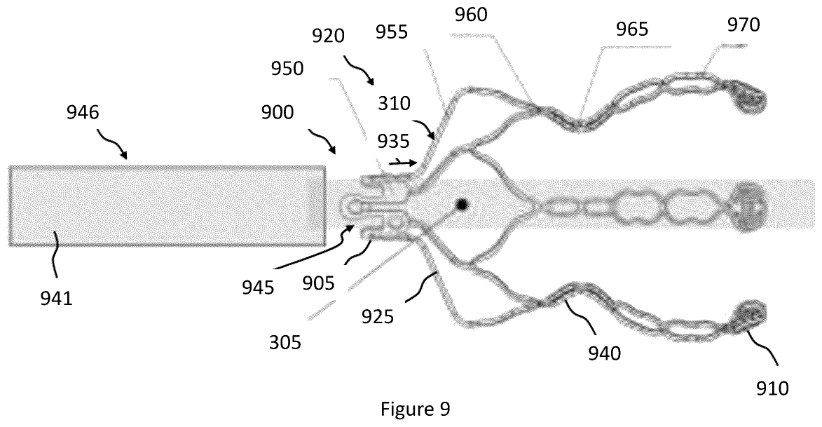

FIG. 9 shows a schematic side view of an implant system 900 with an implant 305 and a fastening device 310 for fastening the implant 305 in a channel according to a design example.

The fastening device 310 has a coupling section 905, a fastening section 910 and a transfer section 925. The coupling section 905 is shaped for coupling with the implant 305. The fastening section 910 is configured to contact an inside of the vascular canal in a fixture position 920 of the fastening device 310 shown here to anchor the implant 305 in the vascular canal. The transfer section 925 connects the coupling section 905 to the fastening section 910 and is shaped to cause the fastening device 310 to transfer from the fastening position 920 to a release position in which the fastening device 310 releases the implant 305 in response to a push actuation 935.

According to this design example, implant 305 is a pump that is configured for use with or as a cardiac support system according to a design example. According to this example, the implant 305 is configured to be implanted in a blood vessel and can be fixed there in the fastening position 920 shown here by means of the fastening device 310. The blood vessel may be the aorta, for example. The pump is formed according to this design example for conveying a fluid, in this case blood, in the channel and for this purpose has, according to this design example, a rotatable impeller for generating a fluid suction and a drive device for driving the impeller and further may have at least one sensor and sensor cable. According to an alternative design example, the implant 305 is configured as any other object or device that can be implanted in a channel or as another minimally invasive implantable device.

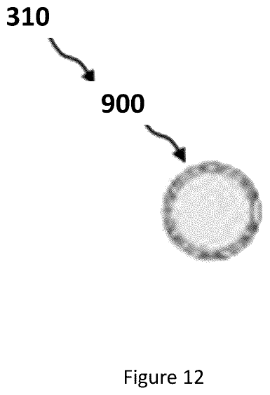

According to this design example, the fastening section 910 is shaped to contact the inside of the vascular canal in the fastening position 920 in such a way that the implant 305 can be anchored non-positively in the vascular canal, here it can be clamped. According to a design example, the fastening section 910 contacts the inside of the canal radially. In the fastening position 920 the fastening device 310 is deployed (e.g., unfolded). According to this design example, in the fastening position 920, the transfer section 925 and/or the fastening section 910 are arranged extending away from the coupling section 905. According to a design example, the fastening device 310 is arranged folded in the release position shown in FIGS. 11 and 12. According to this design example, pressure actuation 935 can be applied to the transfer section 925 by means of a release sleeve 941 or a reinforced hose, also known as a retrieval sheath. In the following, by continuing the pressure actuation 935, the fastening device 310 can be folded in and furthermore the entire implant 305 with the fastening device 310 can be accommodated in the release sleeve 941, see also FIGS. 11 and 12.

According to this design example, the fastening device 310 is formed as a foldable frame and may be in one piece. According to this design example, the coupling section 905 is shaped tubularly to receive the implant 305 in a tubular interior 945 of the coupling section 905 and/or to put the release sleeve 941 over the coupling section 905. According to this design example, the coupling section 905 is mechanically attached to an outer wall of the implant 305, circumferentially according to a design example. According to this design example, a coupling section diameter of the coupling section 905 is thus larger than an implant diameter of the implant 305 and/or smaller than a release sleeve diameter of the release sleeve 941. Furthermore, in the fastening position 920, according to this design example, the coupling section diameter of the coupling section 905 is smaller than a fastening section span of the fastening section 910 and/or a transfer section span of the transfer section 925. In the fastening position 920, according to this design example, the transfer section 925 is arranged to taper radially and/or obliquely between the coupling section 905 and the fastening section 910. According to this example, the span of the transfer section increases from coupling section 905 to fastening section 910. At least one section 905, 910, 925 of the fastening device 310 has a superelastic and/or self-expanding material such as Nitinol according to this design example. According to this design example, at least the transfer section 925 and/or the fastening section 910 have the material comprising a nickel-titanium alloy, for example Nitinol, according to a design example. According to this design example, the fastening device 310 is transferred independently into the fastening position 920 shown here. According to this design example, the fastening section 910 has at least three fastening arms 940, which are shaped to make radial contact with the vascular canal at three contact points in the fastening position 920. According to this design example, the fastening arms 940 have a curved shape that allows elasticity of the fastening section 910.

According to this design example, the coupling section 905 has a component 950 as shown in FIG. 9, wherein component 950 may be made from a laser cut superelastic Nitinol tube having slots that allow the coupling section 905 to radially expand to allow the coupling section to be advanced over the implant 305 and apply inward pressure on the implant 305 via its superelastic properties to remain connected to the implant. Additionally or alternatively, the coupling section 905 may have a mating part (e.g., a protrusion or hole) that mechanically mates with a compatible mating part on the implant 305 to prevent dislodgement of the coupling section from the implant. According to this execution example, the transfer section 925 has a component 955 according to this execution example. Component 955 may be a strut connecting the coupling section 905 to the fastening section 910, wherein the struts 955 protrude radially and is angled distally (i.e., away from the coupling section) when the fastening device 310 is in the fastening position 920, which may facilitate the motion of collapsing the fastening device 310 to the release position 1100 when the release sleave 941 is advanced over the struts 955. The struts 955 may joint to adjacent struts at vertices as shown and may connect to fastening arms 940 at vertices 960. A design example may include a radial pivot section 965 which may be adjacent the vertex 960 and may connect to a distal arm section 970 of the fastening arms 940, wherein the radial pivot sections 965 are configured to elastically allow the distal arm sections 970 to flex in a radial plane toward and away from the central axis yet apply a radially outward force on the vessel canal to anchor the implant in the canal. The radial pivot sections 965 may be more flexible than the distal arm sections 970.

The fastening device 310 presented here, together with the release sleeve 941, can also be described as a release system 946 for releasing an implant 305 which can be fastened in a vascular canal by means of a fastening device 310. The guide sleeve 941 is configured to receive the coupling section 905 and to exert the actuation pressure 935 on the transfer section 925 to transfer the fastening device 310 from the fastening position 920 to the release position 1100 in which the fastening device 310 releases the implant 305 from the vascular canal.

According to this design example, the fastening device 310 presented here realizes a frame which is removable to allow a temporary anchoring of the implant 305 in a blood vessel. In the case of temporary anchoring mechanisms, it is important to make a compromise in terms of the radial force applied to the vascular system. Such a compromise is realized by the fastening device 310 presented here, which on the one hand prevents migration thanks to the fastening device 310, and on the other hand guarantees a safer removal (retrieval) of the implant system 900 without damaging the surrounding tissue. The described frame is advantageously foldable again by pushing on a reinforced tube (e.g., retrieval sheath 941). An inner core, here in the form of implant 305, defines a uniform folding of a preformed Nitinol frame structure of the frame. FIG. 9 shows how the frame is opened: The frame may deploy by retracting the retrieval sheath 941 and anchors the implant 305 within a vascular structure according to a design example.

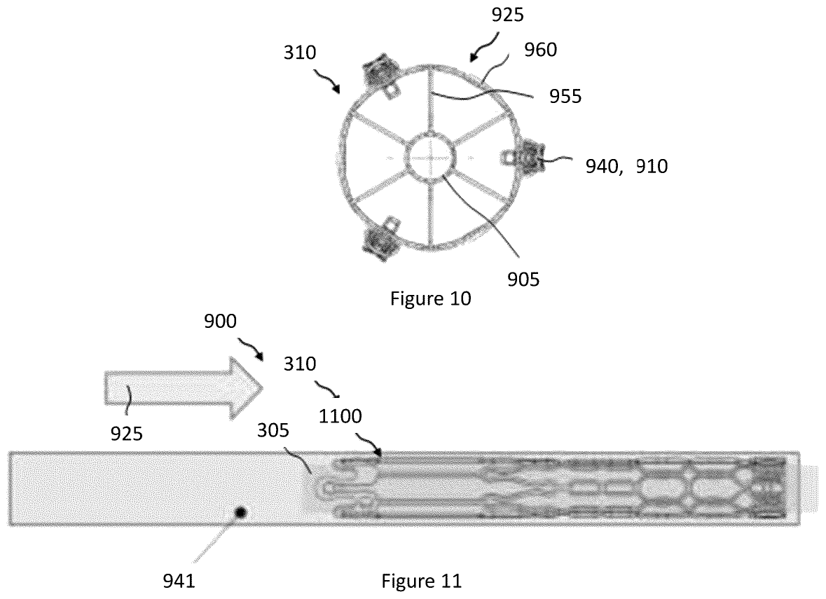

FIG. 10 shows a schematic diagram of a fastening device 310 according to a design example. This can be the fastening device 310 described in the side view of FIG. 9 in the fastening position 920, whereby the fastening device 310 is shown rotated by 90° (i.e., a front view) to the viewer according to this design example.

According to this design example, six equally spaced struts 955 of the transfer section 925 extend obliquely away from the coupling section 905 and/or terminate according to this design example in a braid 1005 of the transfer section 925 which connects the struts 955 in a lattice-like manner. According to this design example, the three fastening arms 940 each extend according to this design example each equally spaced away from a vertex 960 of the lattice, the vertices 960 each being arranged centrally between two adjacent struts 955.

FIG. 11 shows a schematic side view of an implant system 900 according to a design example. This can be the implant system 900 described in FIG. 9, with the difference that the fastening device 310 is arranged in the release position 1100 according to this design example.

In the release position 1100, the fastening device 310 is arranged folded up according to this design example, whereby according to this design example the transfer section and/or the fastening section are arranged adjacent to or pressed against the implant 305. In the release position 1100, the fastening device 310 is arranged according to this design example so as not to contact the vascular canal. In this case the fastening device 310 is arranged according to this design example closely adjacent to the implant 305, according to this design example completely folded in by the guide sleeve 941.

The frame is closed according to this design example by advancing the guide sleeve 941, here in the form of a tube (retrieval sheath), the frame is evenly folded onto the implant 305.

FIG. 12 shows a schematic diagram of a fastening device 310 according to a design example. This may be the fastening device 310 described in the side view of FIG. 11 in the release position 1100, whereby the fastening device 310 is shown rotated by 90°(i.e., front view) to the viewer according to this design example. A maximum diameter of the fastening device 310 in the release position 1100 according to this design example essentially corresponds to the coupling section diameter of the coupling section 905.

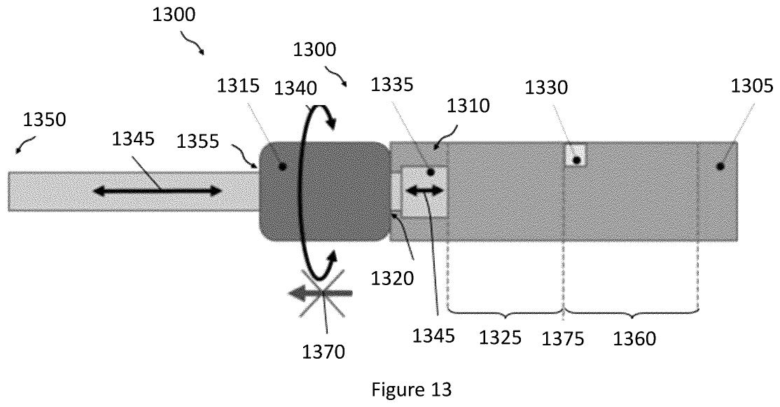

FIG. 13 shows a cross-sectional view of an actuator 1300 for implanting an implant in a vascular canal according to a design example.

The actuator 1300 is shaped according to this design example to implant the implant in the form of a pump shaped according to a design example for use with or as a cardiac support system. According to an example, the vascular canal is a blood vessel into which the pump is configured to be inserted and optionally also to be fastened. The pump is formed according to this design example for conveying a fluid, for example blood, in the channel and for this purpose, according to a design example, has a rotatable impeller for generating a fluid suction and/or a drive device for driving the impeller.

The actuating device 1300 has a housing 1305, a slider device 1310 and an actuating element 1315. The housing 1305 has a housing opening 1320, a setting range 1325 adjacent to the housing opening 1320 and a movable stop device 1330 adjacent to the setting range 1325 on an opposite side of the housing opening 1320. The slider device 1310 has a slider section 1335 which is or can be linearly movably accommodated in the housing 1305. The actuating member 1315 is adapted to cause a linear movement 1345 of the slider means 1310 in the actuating range 1325 in response to an actuation 1340, wherein the stop 1330 is adapted to prevent the linear movement 1345 when the slider portion 1335 contacts the stop 1330 at a contact position in the actuating range 1325 to prevent the slider portion 1335 from passing out of the actuating range 1325.

The actuator 1300 is configured to implant the implant in the vascular canal, according to a design example, by actuating 1340 or different actuations 1340 of the actuator 1300 to move the implant coupled to the actuator 1300 to a desired position in the vascular canal and/or to release or fix it in position.

The actuating device 1300 is configured to be operated according to this design example for manual actuation 1340. According to this design example, the slider section 1335 is realized as an end section of the slider device 1310. A further slider section 1350 of the slider device 1310 opposite to the slider section 1335 is configured for coupling with the implant according to this design example and/or is arranged to be led out of the housing opening 1320. According to an alternative design example, the slider device 1310 has only the slider section 1335, which is coupled with a connecting device which is configured to be coupled to the implant on a side opposite the slider section 1335.

According to this design example, the actuating element 1315 is mechanically coupled to the slider device 1310 and/or arranged outside the housing 1305. An additional slider section 1355 of the slider device 1310 adjoining the slider section 1335 is, according to this design example, led out of the housing opening 1320, passed through the actuating element 1315 and/or arranged mechanically connected to the actuating element 1315. The actuating element 1315 may be shaped as a rotary knob according to this design example, whereby the actuation 1340 can be realized as a rotary actuation on the rotary knob. According to this design example, the actuating element 1315 is configured to affect, in response to an actuation opposite to the actuation 1340, a linear return movement of the slider device 1310 opposite to the slider linear movement 1345 in the setting range 1325.

According to this design example, the housing 1305 also has a second setting range 1360, with the stop device 1330 being located between the setting range 1325 and the second setting range 1360. According to this design example, the slider device 1310 is prevented from passing into the second setting range 1360 by the stop device 1330. According to this design example, the actuating device 1300 comprises at least one release unit adapted to open the stop 1330 in response to a release movement 1370 when the slider portion 1335 is disposed in the contact position, to allow passage of the slider means 1310 into the second actuating range 1360. According to this design example, the slider section 1335 is located in a rest area between the housing opening 1320 and the setting range 1325 or in the setting range 1325. According to this design example, the enabling movement 1370 may be blocked in the positioning of the slider section 1335 shown here.

The actuating device 1300 presented here realizes a handle design and a handle mechanism for a delivery system.

Implants are already being implanted in humans via catheters. This can be done through various access routes. Among others, the “transfemoral”, “transaortal” and “transapical” routes known from the “TAVI” procedures (Transcatheter Aortic Valve Implantation, heart valve replacement) should be mentioned here. An access route is selected depending on the patient (e.g., size, vascular condition, implant size) and the implant. There are special catheter systems for all access routes as well as for implants such as heart valves in order to be able to perform the implantation as precisely and safely as possible. The design and function of the handle as well as the entire delivery system are adapted to the individual steps required as well as the size of the implants. Today's “VAD” (Ventricular Assist Device) systems, or cardiac support systems, are conventionally implanted surgically due to their size and nature without using a delivery system. Short-term cardiac support systems are also delivered either completely without a delivery system along a wire or with simple delivery systems via the transfemoral route.

The Actuator 1300 presented here is an advantageous option for the delivery of a VAD system via a transfemoral, transaortic and transapical approach.

The described handle functional concept of the Actuator 1300 allows a one-handed operation of a complex implant-release mechanism to improve operability and, due to the one-handedness, increased safety. The Actuator 1300 enables the simple and obviously separate controllability of two different setting ranges 1325, 1360, which can also be called “functional ranges”, see also the description of the following figures. A so-called “Point of Thinking” 1375 represents a transitional area between the two functional areas/positioning ranges 1325, 1360. In the operating range 1325, reversible operation is possible according to this design example. As soon as you switch to the second control range 1360, a transition to control range 1325 is no longer possible according to this example. The transition range “Point of Thinking” 1375 requires the user to “consciously” enable it. This transition range is made possible by a functional and mechanical handle design and protects the user from incorrect operation. The advantage of the actuating device 1300 presented here is that ergonomics directly interact with function. Furthermore, operability is improved and the probability of incorrect operation is very low.

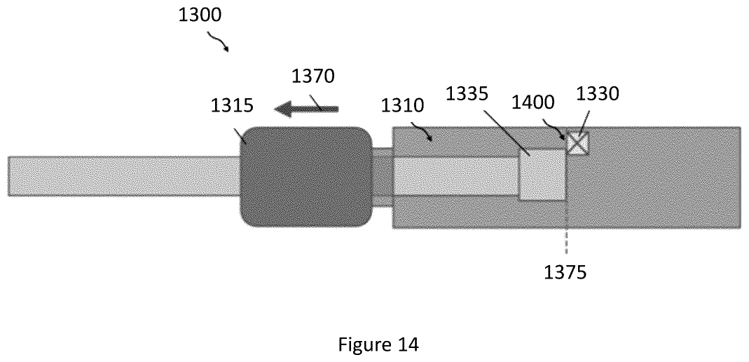

FIG. 14 shows a cross-sectional view of an actuator 1300 for implanting an implant in a vascular canal according to a design example. This may be the actuator 1300 described in FIG. 13, in which the slider device 1310 has performed the linear movement of the slider by actuating the actuator 1315 until the slider section 1335 is moved to the contact position 1400. In the contact position 1400 of the slider section 1335 shown here, the enabling movement 1370 has also been performed and the stop device 1330 has been opened, allowing the slider device 1310 to pass into the second setting range.

According to this design example, the release device was actuated by means of actuating element 1315. According to this design example, the release device is configured to open the stop device 1330 in response to the release movement 1370, which is configured as an actuating element linear movement of the actuating element 1315, in particular a pulling out of the actuating element 1315, when the slider section 1335 is arranged in the contact position 1400. When “pulling out”, the actuating element 1315 was pulled away from the housing according to this design example.

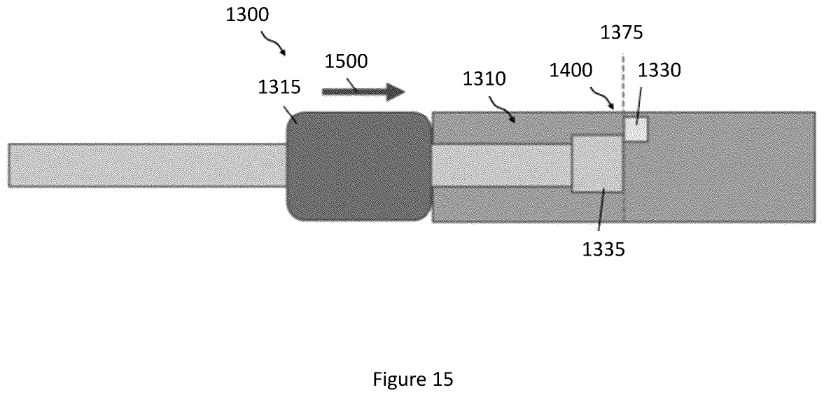

FIG. 15 shows a cross-sectional view of an actuator 1300 for implanting an implant in a vascular canal according to a design example. This may be the actuator 1300 described in the previous figures, in which the slider section 1335 is arranged in the contact position 1400, but in which a blocking movement 1500 has been executed instead of the release movement, which prevents the passage of the slider section 1310 into the second setting range.

According to this design example, the release device is configured to close or keep closed the stop device 1330 in response to the blocking movement 1500 when the slider section 1330 is arranged in the contact position 200 in order to prevent the slider section 1310 from passing into the second setting range 1360.

According to this execution example, the blocking movement 1500 was executed as a movement opposite to the release movement shown in FIG. 14. According to this design example, the blocking movement 1500 represents an actuating element linear movement of the actuating element 1315 opposite to the actuating element linear movement, according to this design example a pushing movement of the actuating element 1315 towards the housing or to the housing.

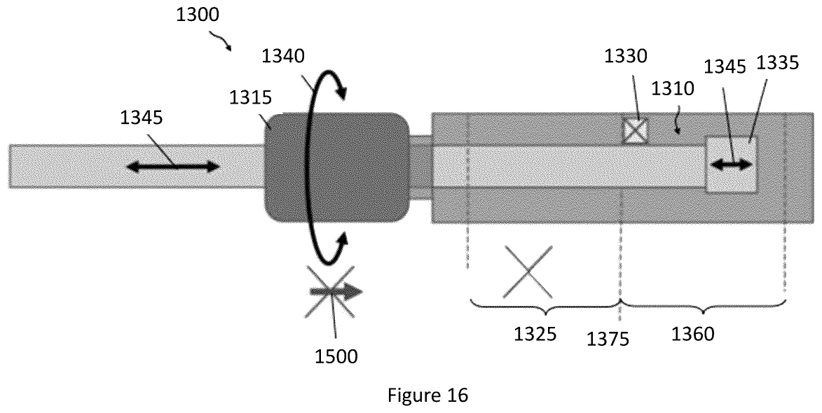

FIG. 16 shows a cross-sectional view of an actuator 1300 for implanting an implant in a vascular canal according to a design example. This may be the actuator 1300 described in the previous FIGS. 13 to 15, in which the slider device 1310 has been transferred to the second setting range 1360 by actuation 1340, release movement and renewed actuation 1340.

According to this design example, the actuating element 1315 is configured to continue the linear movement 1345 in the second range 1360 in response to the actuation 1340. According to a design example, the actuating element 1315 is configured to cause the opposite linear return movement of the slider device 1310 in the second setting range 1360 in response to the opposite actuation.

According to this design example, the release device is configured to close the stop device 1330 as soon as the slider device 1310 is positioned in the second setting range 1360 in order to prevent the slider section 1335 from passing back into the setting range 1325. A performance of the blocking movement 300 is blocked according to this execution example as soon as the enabling movement has been performed and/or the slider device 1310 is arranged in the second positioning range 1360.

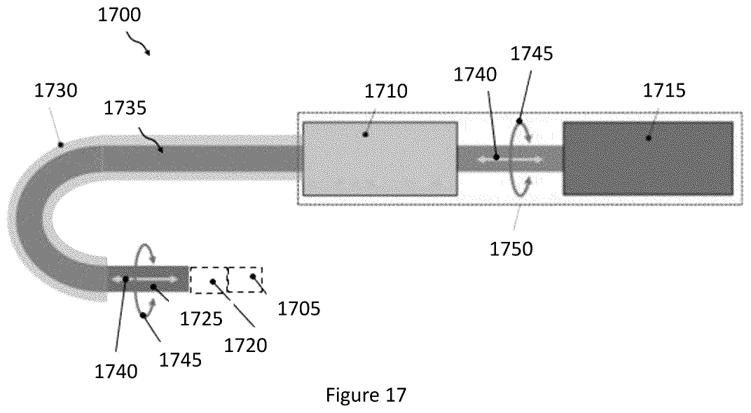

FIG. 17 shows a schematic representation of an actuator 1700 for implanting an implant 1705 in a vascular canal according to a design example.

The actuator 1700 has a guiding device 1710 and a catheter system 1715. The catheter system 1715 is coupled or can be coupled to the guiding device 1710 and comprises a release device 1720 for implanting the implant 1705 and a flexible movement device 1725, which is movable in relation to the guiding device 1710, for moving the release device 1720. The guiding device 1710 is configured to mechanically control a bending operation of the moving device 1725.

The actuator 1700 is shaped according to this design example to implant the implant 1705 in the form of a pump shaped according to a design example for use with or as a cardiac support system. According to an example, the channel is a blood vessel into which the pump is configured to be inserted and optionally also to be fastened. The pump is formed according to this design example for conveying a fluid, for example blood, in the channel and for this purpose, according to a design example, has a rotatable impeller for generating a fluid suction and/or a drive device for driving the impeller. According to an alternative design example, the implant 1705 is configured as any other object or device that can be implanted into a channel or as another minimally invasive implantable device.

According to this design example, the guide device 1710 comprises a bendable outer guide unit 1730, in or on which an inner section 1735 of the moving device 1725 is received or receivable in order to bend the inner section 1735 in response to a bending movement of the outer guide unit 1730 caused by the guide device 1710. According to this design example, the outer guide unit 1730 is tubular and/or formed of a flexible material. According to this design example, the moving device 1725 is configured to be linearly movable and/or rotatable. A linear movement 1740 or rotation 1745 of the movement device 1725 is performed according to an example in response to one or different operating procedures of an operating element of the catheter system 1715 formed for this purpose. Furthermore, according to this example, the movement device 1725 is coupled or can be coupled without rotation and/or translation with respect to at least one guide device section of the guide device 1710.

According to this design example, the actuating device 1700 also has a housing 1750, in which a section of the catheter system 1715 and a section of the guiding device 1710 are accommodated. According to one design example, the housing 1750 has at least one manually operated control element for moving the moving device 1725.

The actuator 1700 realizes an extended controllability for a so-called “delivery system”, such as for a delivery system which according to this design example has the actuator 1700 and the implant 1705. A design of the actuator 1700 enables the extended controllability of the catheter system 1715 along complex anatomical vascular courses. A defined insertion and extended controllability/alignment of the implant 1705 to be delivered is advantageous. Furthermore, the use of the actuator 1700 allows independent alignment in a bending plane with combined freedom of rotation and translation.

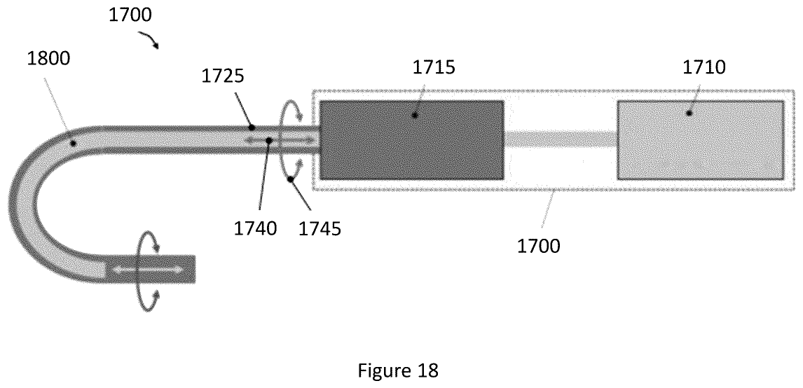

FIG. 18 shows a schematic representation of an actuator 1700 for implanting an implant in a vascular canal according to a design example. This may be the actuator 1700 described in FIG. 17, with the difference that the guide device 1710 according to this design example has instead of the bendable outer guide unit a bendable inner guide unit 1800 which is accommodated at least partially in the movement device 1725 to bend at least a part of the movement device in response to a bending movement of the inner guide unit 1800 caused by the guide device 1710.

According to this design example, the entire movement device 1725, or according to an alternative design example, only the part of the movement device 1725 containing the inner guide unit 1800 is tubular and/or formed from a flexible material.

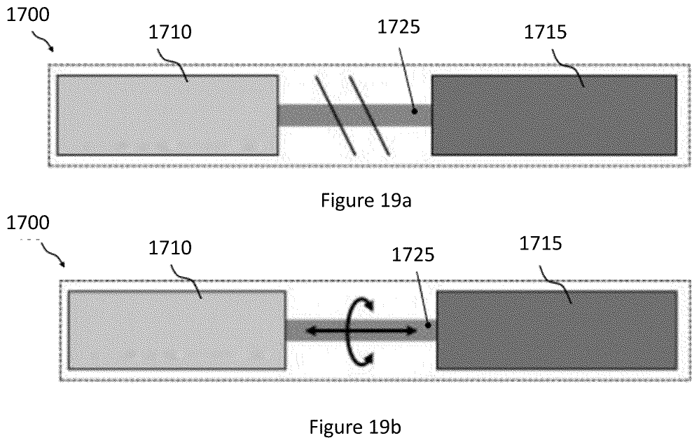

FIG. 19a shows a schematic representation of an actuator 1700 for implanting an implant in a vascular canal according to a design example. This may be one of the actuating devices 1700 described in one of the previous FIGS. 17 to 18.

The catheter system 1715 and/or the guiding device 1710 is configured according to this design example to block a mobility of the movement device 1725. The catheter system 1715 and/or the guiding device 1710 is configured according to this example to block the mobility in response to a blocking signal or a blocking actuation, for example at the control element of the actuating device 1700.

A relative movement of catheter system 1715 and guiding device 1710 is blocked according to this design example.

FIG. 19b shows a schematic representation of an actuator 1700 for implanting an implant in a vascular canal according to a design example. This may be one of the actuating devices 1700 described in one of the previous FIGS. 17 to 19a.

The catheter system 1715 and/or the guiding device 1710 is configured, according to this design example, to release or cause a linear motion and/or rotational motion of the movement device 1725. The catheter system 1715 and/or the guiding device 1710 is configured in accordance with this design example in order to release or cause the linear and/or rotational mobility in response to an enabling signal or an enabling actuation of the actuating device 1700, for example on the control element.

A relative movement of catheter system 1715 and guiding device 1710 is made according to this design example with regard to translation and rotation.

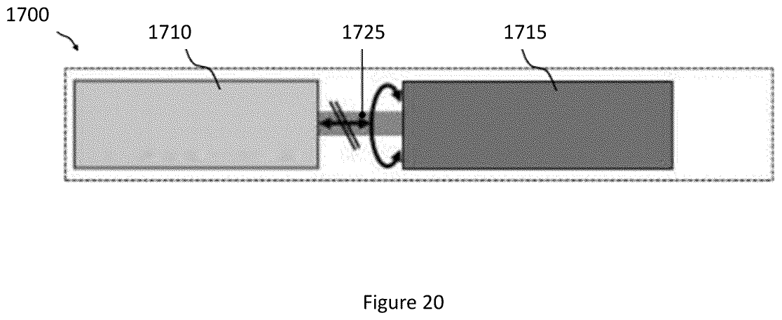

FIG. 20 shows a schematic representation of an actuator 1700 for implanting an implant in a vascular canal according to a design example. This may be one of the actuating devices 1700 described in one of the previous FIGS. 17 to 19b.

The catheter system 1715 and/or the guiding device 1710 is configured according to this design example to block a linear movement of the moving device 1725 and/or to release or cause a rotational movement of the moving device 1725. The catheter system 1715 and/or the guiding device 1710 is configured according to this design example to block the linear mobility of the moving device in response to a control signal or a control actuation, for example at the control element. Additionally or alternatively, the catheter system 1715 and/or the guiding device 1710 is configured in accordance with this design example to enable or cause the rotational mobility of the moving device 1725 in response to the control signal or the control actuation or in response to a further control signal or a further control actuation, for example at the operating element.

A relative movement of catheter system 1715 and guiding device 1710 is blocked according to this design example with respect to a translation in any position, with free rotation.

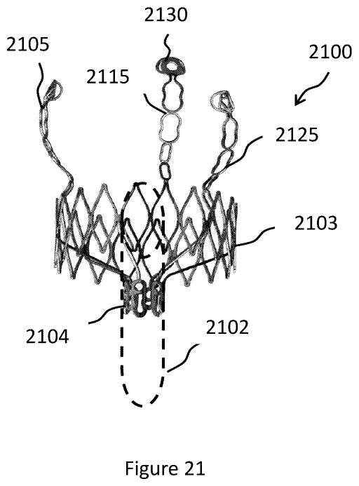

FIG. 21 shows a schematic representation of a design example of a holding frame 2100 for a cardiac support system 2102, which is only shown schematically and therefore dashed in FIG. 21.

The holding frame 2100 is configured as a wire mesh element. In the illustration in FIG. 21, the Cardiac Support System 2102 is also only schematic and therefore dashed whereby, according to the example shown in this design example, a ring-shaped lattice element 2103 is provided here, which, for example, lies against an aortic wall in the implanted state. The lattice element 2103 is coupled to the body of the Cardiac Support System 2102 (for example, an motor block) via retaining struts 2104 and thus allows positioning at a desired location of the Cardiac Support System 2102 in the aorta. The support struts 2104 can, for example, form a tube that is placed or clamped around an outer surface of the Cardiac Support System 2102 and thereby clamp on to the Cardiac Support System 2102. The holding frame 2100 may have three or more feet 2105, 2115, 2125 for Placement of the Cardiac Support System 2102 in an aorta, wherein said feet 2105, 2115 and 2125 are configured as interconnected wire loops and extend away from the lattice element 2103 on a side opposite the support struts 2104.

Furthermore, the holding frame 2100 has an X-ray marker 2130 which is formed, to enable the position of at least one of the feet 2105, 2115, 2125 in the aorta to be checked using X-rays. The X-ray marker 2130 may be located on a foot 2115 of the holding frame 2100 and has a structure that differs from that of an X-ray image of another component of the holding frame 2100. The holding frame 2100 and the feet 2105, 2115, 2125 are configured as a lattice structure. The feet 2105, 2115, 2125 of the holding frame 2100 may be configured to position the holding frame 2100 between the leaflets of an aortic valve. The holding frame 2100 comprises at least partially a Nitinol material. The holding frame 2100 may be centrally and permanently connected to the Cardiac Support System 2102. The holding frame 2100, with which the implant is fixed inside the aorta, may consist of a Nitinol braid or laser cut Nitinol tube. The Cardiac Support System 2102 may be connected centrally and permanently to the holding frame 2100, for example, by means of the holding struts 2104. The holding frame 2100 can also be referred to as a Nitinol frame. The three feet 2105, 2115, 2125 may facilitate the holding frame 2100 to be positioned between the three valve leaflets of the aortic valve. This helps to improve function of the Cardiac Support System 2102.

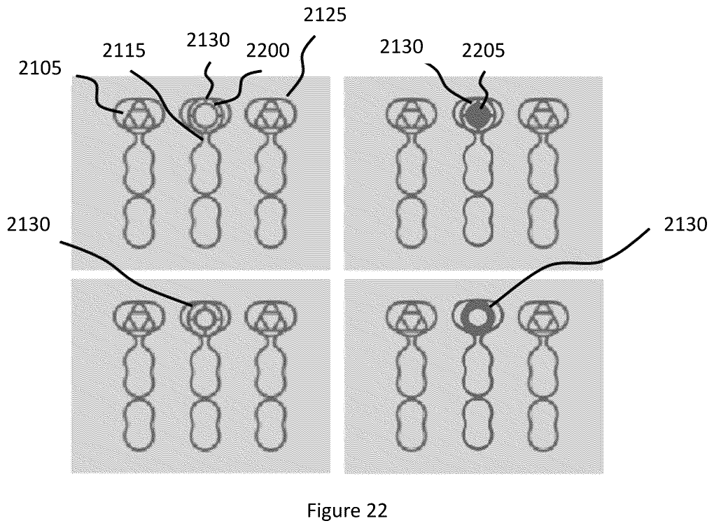

FIG. 22 shows a schematic representation of an example of an X-ray marker 2130 of a holding frame for a Heart support system 2102, which may be the holding frame for a heart support system 2102 described in FIG. 21. FIG. 22 shows a total of four examples of an X-ray marker 2130, which is mounted on a base 2115 of the holding frame or embedded in the base 2115 and has a design that is different from the one shown in an X-ray image of another component of the holding frame. The positioning of the holding frame with the foot 2115 is performed during the operation and during a follow-up check with the aid of imaging techniques using X-rays, so that the position of the X-ray marker 2130 can be clearly identified by the differing structure, so that it is also possible to see from this how the holding frame as a whole and thus also the position of the heart support system is positioned. The design of the feet 2105, 2115, 2125 and the X-ray marker 2130, which can be easily distinguished visually in the X-ray image, allows the physician to determine the orientation of the holding frame within the vessel using imaging methods, especially X-rays. In order to guarantee or at least facilitate a simple and clear identification of the orientation, at least one of the three feet 2105, 2115, 2125 stands in strong contrast to the others with respect to its structure. The X-ray marker 2130 of one of the feet 2115 can, for example, be different degrees of filling 2205 (surface-contour), as shown in the partial figure on the upper right. In this partial figure, the X-ray marker 2130 has a high degree of filling, which is clearly visible in the X-ray image. Further differentiation possibilities can be achieved by a distinguishable symbol 2200, by a distinguishable design of the outer contour, by a distinguishable size of the foot 2115 or by a distinguishable pattern can be achieved. For example, the X-ray marker 2130 is configured as a narrow ring in the upper left figure, whereas it is configured as a wide ring in the lower left figure. In the illustration from the lower right figure the X-ray marker is configured as a filled wide ring. In each of the four partial figures it can be seen that the X-ray marker 2130 at foot 2115 has a different shape than the other two feet 2105 and 2125, so that the position of the X-ray marker 2130 in relation to the other feet can be clearly, quickly and reliably identified.

In FIG. 22, the X-ray marker 2130 in a design example is configured as a symbol 2200 that can be distinguished from a further area of the holding frame. In a second example, this symbol 2200 has a distinguishable filling 2205, as described above. The third and the fourth design example shows a further design of the symbol of the X-ray marker 2130.

Compared to conventional structures, the variants of X-ray marker 2130 presented here are unproblematic in terms of production and integration into Nitinol structures and the risk of detachment from their support. By marking the Nitinol frame during the manufacturing process, for example, these risks are avoided and costs are saved.

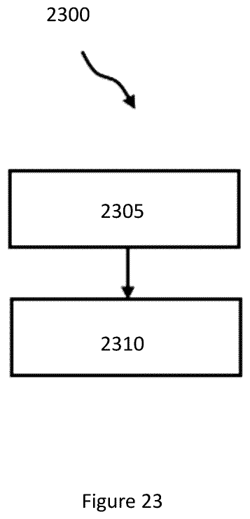

FIG. 23 shows a flow chart of a procedure 2300 for manufacturing an implant device according to a design example. This may be one of the implant devices described in any of FIGS. 3 to 22.

Procedure 2300 has a step 2305 of provisioning and a step 2310 of coupling. The provision step 2305 provides an implant for implantation into a vascular canal and a fastening device connected (e.g. releasably connected) to the implant, which is movable between a fastening position in which the fastening device fastens or makes fastenable the implant in the vascular canal and a release position in which the fastening device releases the implant. In step 2310 of the coupling, a transfer device is coupled to the fastening device, the transfer device being adapted to cause transfer of the fastening device between the fastening position and the release position in response to actuation.

The steps of the procedure presented here can be repeated as well as performed in a different order than described.

### Numbered Example Embodiments

The following is a non-exhaustive list of numbered example embodiments (NEE).
NEE 1. An implant device comprising: an implant for implanting in a canal; a fastening device connected to the implant and movable between a fastening position, in which the fastening device fastens or makes the implant fastenable in the channel, and a release position, in which the fastening device releases the implant; and a transfer device coupled to the fastening device and adapted to cause the fastening device to be transferred between the fastening position and the release position in response to an actuation.
NEE 2. The implant device of Numbered Example Embodiment (NEE) 1, in which the fastening device is formed as a foldable frame, the foldable frame being arranged unfolded in the fastening position and folded in the release position.
NEE 3. The implant device of NEE 2, in which the transfer device has at least one pull cord which is formed for the purpose of causing, in response to actuation in the form of a pulling movement, the transfer of the fastening device from the fastening position into the release position and, in response to a further actuation in the form of a release movement, the transfer of the fastening device from the release position into the fastening position.
NEE 4. The implant device according to NEE 3, in which the pulling cable has a pulling cable driver which is shaped for coupling to an operating device for operating the transfer device.
NEE 5. An operating device for operating a transfer device of the implant device of NEE 1, wherein the operating device is designed so as to be couplable to the transfer device and, in response to an operation, causes the actuation of the transfer device.
NEE 6. The operating device according to NEE 5, which has a runner device which is formed such that it can be coupled to a pull cord driver of a pull cord of the transfer device.
NEE 7. The operating device of NEE 6, adapted to cause actuation in a coupled state with the transfer device in response to an operator formed as a rotary motion.
NEE 8. The operating device of NEE 6, comprising a supporting extrusion means for receiving at least a portion of the transfer means to resist a compressive force during operation.
NEE 9. An implant system with the implant device of NEE 1 and an operating device of NEE 5.
NEE 10. A method for manufacturing the implant device of NEE 1, said method comprising: providing an implant for implantation into a canal and a fastening device connected to the implant, the fastening device being movable between a fastening position, in which the fastening device makes the implant fastenable or fixable in the canal, and a release position, in which the fastening device releases the implant; and coupling a transfer device to the fastener, the transfer device being adapted to cause transfer of the fastener between the fastening position and the release position in response to an actuation.
NEE 11. Apparatus arranged to execute and control the steps of the process according to NEE 10 in corresponding units.
NEE 12. A computer program adapted to execute and control the steps of the method according to NEE 10.
NEE 13. A machine-readable storage medium on which the computer program according to NEE 12 is stored.
NEE 14. The implant device of NEE 1 in which the transfer device comprises: a sling shaped to wrap around the fastening device in a fastening position in which the fastening device fastens or makes fastenable the implant in the channel; and an actuating device which is designed to cause the sling to be tightened when the transfer device is looped around the fastening device in order to transfer the fastening device from the fastening position to a release position in which the fastening device releases the implant.
NEE 15. The implant device of NEE 14, in which the actuating device is shaped to cause the sling to be tightened in response to a rotary actuation and/or sliding actuation and/or pushing actuation.
NEE 16. The transfer device of NEE 15, in which the actuating device is adapted to cause the sling to open or extend and wrap the fastening device by means of the sling in response to a preceding rotary actuation, preceding sliding actuation, or preceding pushing actuation.
NEE 17. The transfer device of NEE 16, comprising an actuating member adapted to receive the rotary actuation and/or sliding actuation and/or pushing actuation and/or preceding rotary actuation and/or preceding sliding actuation and/or preceding pushing actuation.
NEE 18. The transfer device of NEE 17, comprising a sleeve device shaped to receive the fastening device in the release position and the implant.
NEE 19. The transfer device according to NEE 18, in which the sleeve device comprises a sliding device which is arranged or arrangeable in the sleeve device and is designed to resist a force in the longitudinal direction of the sleeve device.
NEE 20. The transfer device according to NEE 18, in which the actuating device is designed to cause the sleeve device to be extended via the fastening device in the release position and the implant in response to a further rotary actuation, further sliding actuation, or further pressure actuation.
NEE 21. An implant system with a transfer device according to NEE 20 and the implant with the fastening device.
NEE 22. The implant system according to NEE 21, in which the fastening device is formed as a collapsible frame, the frame being arranged collapsed in the release position and deployed in the fastening position.
NEE 23. The Implant system according to NEE 22, in which the implant has a shaft which is shaped to guide the release device and/or which is guided through a loop of the sling and/or which runs along a guide rod of the sling.
NEE 24. A method for manufacturing the release device according to NEE 14, the method comprising: providing a loop shaped to wrap around the fastening device in a fastening position in which the fastening device fastens or makes fastenable the implant in the canal; and coupling an actuating device to the loop in order to produce the release device, the actuating device being designed to cause the loop to be tightened in the coupled state with the loop when the fastening device is wrapped around it in order to transfer the fastening device from the fastening position into a release position in which the fastening device releases the implant.
NEE 25. An apparatus arranged to execute and control the steps of the process according to NEE 24 in corresponding units.
NEE 26. A computer program adapted to execute and control the steps of the method according to NEE 24.
NEE 27. Machine readable storage medium on which the computer program according to NEE 26 is stored.
NEE 28. The fastening device of the implant device of NEE 1, the fastening device having the following features: a coupling portion shaped for coupling to the implant, a fastening portion shaped to contact an inner side of the channel in a fastening position of the fastening device for fastening the implant in the channel, and a transfer portion connecting the coupling portion to the attachment portion and configured to cause transfer of the attachment device from the attachment position to a release position in response to a push actuation, in which the attachment device releases the implant.
NEE 29. The fastening device according to NEE 28, in which the coupling section is formed in a tubular shape for receiving the implant in a tubular interior of the coupling section and for advancing a release sleeve over the coupling section.
NEE 30. The fastening device of NEE 29, in which in the fastening position a coupling section diameter of the coupling section is smaller than a fastening section span of the fastening section and a transfer section span of the transfer section.
NEE 31. The fastening device of NEE 25, in which, in the fastening position, the transfer section is arranged radially and obliquely between the coupling section and the fastening section.
NEE 32. The fastening device according to NEE 31, wherein at least a portion of the fastening device comprises a superelastic, shape memory and/or self-expanding material.
NEE 33. The fastening device according to NEE 32, which is formed as a collapsible frame, optionally in one piece.
NEE 34. The fastening device of NEE 28, wherein the fastening portion comprises at least three fastening arms shaped to radially contact the duct at at least three contact points in the fastening position.
NEE 35. An implant system having a fastening device of NEE 35 and the implant being coupled or couplable to the fastening device.
NEE 36. A release system for releasing an implant which can be fastened in a channel by means of a fastening device, wherein the release system comprises the fastening device according to NEE 28 and a release sleeve formed therefor, to receive the coupling portion and to apply the pressure actuation to the transfer portion to transfer the fastening device from the fastening position to the release position in which the fastening device releases the implant.
NEE 37. A method for manufacturing an implant system according to NEE 31, the method comprising the following steps: providing the implant and the fixation device; and coupling the fastening device to the implant such that the coupling portion is coupled to the implant and the transfer portion is arranged such that the fastening device is transferred from the fastening position to the release position in response to a pressure actuation on the transfer portion.
NEE 38. An actuator for implanting an implant in a canal, the actuator comprising the following features: a housing having a housing opening, an adjustment region adjacent to the housing opening, and a movable stop device adjacent to the adjustment region on an opposite side of the housing opening; a slider means, wherein a slider portion of the slider means is received or receivable in the housing in a linearly movable manner; an actuating member adapted to cause a linear movement of the slider means in the actuating range in response to an actuation, wherein the stop means is formed therefor, to prevent the linear movement of the slider when the slider portion contacts the stop means at a contact position in the actuating range to prevent the slider portion from passing out of the actuating range.
NEE 39. The actuating device according to NEE 38, in which the housing has a second actuating region, wherein the stop device is arranged between the actuating region and the second actuating region.
NEE 40. The actuating device according to NEE 39, comprising release means adapted to open the stop means in response to a release movement when the slider portion is disposed in the contact position to allow passage of the slider means into the second actuating region.
NEE 41. The actuating device according to NEE 40, in which the release device is designed to open the stop device in response to the release movement, which is designed as an actuating element linear movement of the actuating element, in particular pulling out the actuating element, when the slider section is arranged in the contact position.
NEE 42. The actuating device according to one of NEE 40, in which the release means is adapted to close the stop means in response to a blocking movement when the slider portion is arranged in the contact position to prevent the slider means from passing into the second actuating range.
NEE 43. The actuating device according to NEE 42, in which in the release device the locking movement represents a movement opposite to the release movement.
NEE 44. The actuating device according to NEE 40, wherein the release means is adapted to close the stop means once the slider means is positioned in the second actuating area to prevent the slider portion from passing back into the actuating area.
NEE 45. The actuating device according to NEE 44, in which the release device is actuatable by means of the actuating element.
NEE 46. The actuating device of NEE 45, in which the actuating member is adapted to cause, in response to an actuation opposite to the actuation, a linear return movement of the slider device opposite to the linear movement of the slider in the actuating range.
NEE 47. The actuating device according to NEE 38, in which the actuating element is formed as a rotary knob, in particular wherein the actuating element is a rotary actuating element.
NEE 48. A method for manufacturing the actuator of NEE 38, the method comprising the steps of: providing a housing having a housing opening, an adjustment region adjacent to the housing opening and a movable stop device adjacent to the adjustment region on an opposite side of the housing opening, a slider device and an actuating element; and mounting the housing, the armature device and the actuator such that an armature portion of the armature device is received in the housing in a linearly movable manner, and that the actuator is arranged to cause an armature linear movement of the armature device in the actuating area in response to an actuation, wherein the stop means is arranged to prevent the linear movement of the slider when the slider portion contacts the stop means at a contact position in the actuating area to prevent the slider portion from passing out of the actuating area.
NEE 49. An apparatus arranged to execute and/or control the steps of the method according to NEE 48 in corresponding units.
NEE 50. A computer program adapted to execute and/or control the steps of the method according to NEE 48.
NEE 51. Machine readable storage medium on which the computer program according to NEE 50 is stored.
NEE 52. An actuator for implanting an implant in a canal, the actuator comprising the following features: a guiding device, and a catheter system coupled or adapted to be coupled to the guiding device, having a release device for implanting the implant and a flexible movement device, which is movable and movable with respect to at least part of the guide device, for moving the release device, wherein said guiding device is adapted to control a bending operation of said release device.
NEE 53. The actuator according to NEE 52, wherein the guide device comprises a bendable outer guide unit in or on which an inner portion of the movement device is received or receivable to bend the inner portion in response to a bending movement of the outer guide unit caused by the guiding device.
NEE 54. The actuator according to NEE 52, wherein the guiding device comprises a bendable inner guide unit received or receivable in a portion of the movement device to bend the portion in response to bending movement of the inner guide unit caused by the guiding device.
NEE 55. The actuator according to NEE 54, in which the movement device is designed to be linearly movable and/or capable of rotation.
NEE 56. The actuator according to NEE 55, in which the movement device is coupled or can be coupled without rotation and/or translation with respect to at least one guide device section of the guiding device.
NEE 57. The actuator according to NEE 54, wherein the catheter system and/or the actuator is adapted to block a mobility of the movement device.
NEE 58. The actuator according to NEE 57, in which the catheter system and/or the guiding device is designed to release or cause a linear movement and/or rotational movement of the movement device.
NEE 59. The actuator according to NEE 58, in which the catheter system and/or the guiding device is designed to block a linear movement of the moving device and/or to release or cause a rotational movement of the movement device.
NEE 60. A method for manufacturing an actuator according to NEE 52, the method comprising the steps of: providing a guiding device and a catheter system having a release device for implanting the implant and a bendable movement device for moving at least a portion of the release device; and coupling the guide device and the catheter system such that the movement device is movable with respect to the guide device and that the guide device is arranged to control a bending operation of the movement device to produce the actuator.
NEE 61. An apparatus arranged to execute and control the steps of the method according to NEE 60 in corresponding units.
NEE 62. A computer program adapted to execute and control the steps of the method according to NEE 60.
NEE 63. Machine readable storage medium on which the computer program according to NEE 62 is stored.
NEE 64. A support frame for a cardiac assist system, the support frame having the following features: at least two feet for placing the cardiac assist system in an aorta; at least one X-ray marker adapted to enable the position of at least one of the feet in the aorta to be checked using X-rays.
NEE 65. The holding frame according to NEE 64, wherein the X-ray marker is arranged on at least one foot of the holding frame, in particular wherein the X-ray marker has a structure different from that in an X-ray image of another component of the holding frame.
NEE 66. The holding frame according to NEE 65, wherein the holding frame and/or the feet are formed as a lattice structure.
NEE 67. The holding frame according to NEE 65, wherein the X-ray marker has a different filling structure than another area of the holding frame.
NEE 68. The holding frame according to NEE 67, wherein the X-ray marker forms a symbol which is distinguishable from a further region of the holding frame.
NEE 69. The holding frame according to NEE 64, whereby the X-ray marker has a distinguishable shape of an outer contour of the holding frame.
NEE 70. The holding frame according to NEE 69, wherein the X-ray marker has a size which is distinguishable from a further region of the holding frame.
NEE 71. The holding frame according to NEE 70, whereby the X-ray marker has a distinguishable pattern of the holding frame.
NEE 72. The holding frame according to NEE 64, wherein the two feet of the support frame are configured to position the support frame between leaflets of an aortic valve.
NEE 73. The holding frame according to NEE 72, wherein the support frame comprises at least partially a Nitinol material.
NEE 74. The cardiac assist system with a holding frame according to NEE 64, wherein the holding frame is centrally and/or permanently connected to the cardiac assist system.
NEE 75. A method for manufacturing a holding frame according to any of the foregoing NEEs 64 to 74, the method comprising the following step: forming a holding frame with at least two feet for placing the cardiac assist system in an aorta, wherein in the step of forming at least one X-ray marker adapted to allow the position of at least one of the feet in the aorta to be checked using X-rays.
NEE 76. A computer program adapted to execute and control the step of the method according to NEE 75.
NEE 77. Machine readable storage medium on which the computer program according to NEE 75 is stored.

If an execution example includes an “and/or” link between a first characteristic and a second characteristic, it should be read in such a way that the execution example has both the first characteristic and the second characteristic according to one execution form and either the first characteristic only or the second characteristic only according to another execution form.

Various modifications to the implementations described in this disclosure will be readily apparent to those skilled in the art, and the generic principles defined herein can be applied to other implementations without departing from the spirit or scope of this disclosure. Thus, the disclosure is not intended to be limited to the implementations shown herein, but is to be accorded the widest scope consistent with the claims, the principles and the novel features disclosed herein. The word “example” is used exclusively herein to mean “serving as an example, instance, or illustration.” Any implementation described herein as “example” is not necessarily to be construed as preferred or advantageous over other implementations, unless otherwise stated.

Certain features that are described in this specification in the context of separate implementations also can be implemented in combination in a single implementation. Conversely, various features that are described in the context of a single implementation also can be implemented in multiple implementations separately or in any suitable sub-combination. Moreover, although features can be described above as acting in certain combinations and even initially claimed as such, one or more features from a claimed combination can in some cases be excised from the combination, and the claimed combination can be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. Additionally, other implementations are within the scope of the following claims. In some cases, the actions recited in the claims can be performed in a different order and still achieve desirable results.

It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

What is claimed is:

1. An implant device for use as a heart support system, the implant device comprising:
an implant comprising a heart pump and configured for implanting in a vascular canal;
a fastening device connected to the implant and movable between a fastening position, in which the fastening device is configured to fasten in the vascular canal, and a release position, in which the fastening device is configured to release the implant; and
a transfer device coupled to the fastening device and adapted to cause the fastening device to move between the fastening position and the release position in response to an actuation.

2. The implant device of claim 1, in which the fastening device is formed as a foldable frame, the foldable frame being unfolded in the fastening position and being folded in the release position.

3. The implant device of claim 2, in which the transfer device comprises at least one pull cord, the at least one pull cord configured to cause, in response to actuation in the form of a pulling movement, the transfer of the fastening device from the fastening position into the release position and, in response to a further actuation in the form of a release movement, the transfer of the fastening device from the release position into the fastening position.

4. The implant device of claim 1, wherein the fastening device comprises at least three fastening arms shaped to radially contact the vascular canal in at least three contact points in the fastening position.

5. The implant device of claim 1, wherein the fastening device comprises a holding frame for the implant, the holding frame comprising:
at least two feet for placing the implant in the vascular canal; and
at least one X-ray marker adapted to enable a position of the fastening device in the vascular canal to be checked using X-rays.

6. The implant device of claim 5, wherein the at least one X-ray marker is arranged on at least one foot of the holding frame, and wherein the X-ray marker has a structure different from that in an X-ray image of another component of the holding frame.

7. The implant device of claim 5, wherein the at least two feet of the holding frame are configured to position the holding frame between leaflets of an aortic valve.

8. The implant device of claim 5, wherein the holding frame comprises a Nitinol material.

9. The implant device of claim 1, wherein the transfer device further comprises:
a sling shaped to wrap around the fastening device in the fastening position; and
an actuating device which is configured to cause the sling to be tightened when the transfer device is looped around the fastening device in order to transfer the fastening device from the fastening position to the release position.

10. The implant device of claim 9, wherein the actuating device is adapted to cause the sling to open or extend and wrap the fastening device by means of the sling in response to a preceding rotary actuation, preceding sliding actuation, or preceding pushing actuation.

11. The implant device of claim 1, further comprising a sleeve device shaped to receive the fastening device in the release position and the implant.

12. A system for implanting an implant comprising a heart pump in a vascular canal, the system comprising:
a housing comprising a housing opening, a first actuating region adjacent to the housing opening, and a movable stop device adjacent to the first actuating region on an opposite side of the housing opening;
a slider having a slider portion configured to be received in the housing in a linearly movable manner;
the implant comprising the heart pump connected to the slider; and
an actuating member adapted to cause a linear movement of the slider in response to an actuation, wherein the stop device is configured to prevent the linear movement of the slider when the slider portion contacts the stop at a contact position and prevent the slider portion from exiting the first actuating region.

13. The system of claim 12, wherein the housing further comprises a second actuating region, and wherein the stop device is arranged between the first actuating region and the second actuating region.

14. The system of claim 13, further comprising at least one release unit configured to open in response to a release movement when the slider portion is disposed in the contact position to allow passage of the slider into the second actuating region.

15. The system of claim 14, wherein the at least one release unit is configured to open the stop device in response to the release movement, and wherein the at least one release unit is configured as an actuating element with linear movement of the actuating member, the linear movement comprising pulling out the actuating element, and wherein the slider section is arranged in the contact position.

16. The system of claim 14, wherein the at least one release unit is configured to close the stop device in response to a blocking movement when the slider is arranged in the contact position to prevent the slider from passing into the second actuating region.

17. The system of claim 16, wherein the blocking movement of the release device represents a movement opposite to the release movement.

18. The system of claim 14, wherein the at least one release unit is configured to close the stop device once the slider is positioned in the second actuating region to prevent the slider portion from passing back into the first actuating region.

19. The system of claim 18, wherein the at least one release unit is actuatable by the actuating member, and wherein the actuating member is further configured to cause, in response to an actuation opposite to the actuation, a linear return movement of the slider opposite to the linear movement of the slider in the actuating range.

20. The system of claim 15, wherein the actuating member further comprises a rotary knob, and wherein the actuating element is a rotary actuating element.
